SEQUENCE LISTING (7 sequences)

SEQ ID NO: 1 - DNA, 1329 bp, synthetic construct
  misc_feature 1..1329: note = Synthetic
  source 1..1329: mol_type = other DNA
atggcgccccgaagcctcctcctgctgctctcaggggccctggccctgaccgatacttgggcaggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgccttctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagcctactcagtggtcagcggactcttgatgataacatggtggtcaagcttatttctcctgggggtgctcttccaaggatatttgggctgcctccggagtcacagtgtcttgggccgccggaaggtgggtgacatgtggatcttgttttttttgtggctgtggacatctttcaacactgccttcttggccttgcaaagccttcgctttggcttcggctttaggaggggcaggagcttccttcttcgttcttggcaccatcttatgaaaagggtccagattaagatttttgactag

SEQ ID NO: 2 - DNA, 7611 bp, synthetic construct
  misc_feature 1..7611: note = Synthetic
  source 1..7611: mol_type = other DNA
agcggcccgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggtttttccagtcacacctcaggacctttaagaccaatgacttacaaggcagctgtagatcttagccactttttaaaagaaaagaggggactggaagggctaattcactcccaacgaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattaccctgtgggcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatctaatacgactcactatagggagagagagagaattaccctcactaaagggaggagaagcatgaattctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagacgtcgtttagtgaaccgtcagatcactagatgctttattggatccacaagtttgtacaaaaaagcaggcttgaaggaattcggtaccatggcgccccgaagcctcctcctgctgctctcaggggccctggccctgaccgatacttgggcaggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgccttctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagcctactcagtggtcagcggactcttgatgataacatggtggtcaagcttatttctcctgggggtgctcttccaaggatatttgggctgcctccggagtcacagtgtcttgggccgccggaaggtgggtgacatgtggatcttgttttttttgtggctgtggacatctttcaacactgccttcttggccttgcaaagccttcgctttggcttcggctttaggaggggcaggagcttccttcttcgttcttggcaccatcttatgaaaagggtccagattaagatttttgactagctcgagtgcggccgc

SEQ ID NO: 3 - DNA, 21 bp, synthetic construct
  misc_feature 1..21: note = Synthetic
  source 1..21: mol_type = other DNA
taaagagaactgagggctctg

SEQ ID NO: 4 - DNA, 21 bp, synthetic construct
  misc_feature 1..21: note = Synthetic
  source 1..21: mol_type = other DNA
ggcgtagatgtccgataagaa

SEQ ID NO: 5 - DNA, 21 bp, synthetic construct
  misc_feature 1..21: note = Synthetic
  source 1..21: mol_type = other DNA
taaaggagaaagaagaggagg

SEQ ID NO: 6 - DNA, 21 bp, synthetic construct
  misc_feature 1..21: note = Synthetic
  source 1..21: mol_type = other DNA
gggagaaagaaggaggataaa

SEQ ID NO: 7 - DNA, 321 bp, synthetic construct
  misc_feature 1..321: note = Synthetic
  source 1..321: mol_type = other DNA
cacccaagagcaagtcccgctacattagcgccggaagtatccgcttgacgtccatgggcgcctcctccgcggtcaggctggggttcagtgtcggtctgtagacgacctcacacactctcggcgggcgcgagaccaacatcatcggctcgtccaaggagtccgagtgagccttggtgacgagaaccgggaaccgaaagacacaaagggcgagggttatgaggccggggaggacgaggtaggtgccgcgggcgccgagtatagagcctaagaggcgcagcgacagcttcgcgtgcttgaggagcagcaggtgcatcgggtgga

(19) United States
(12) Patent Application Publication
(10) Pub. No.: US 2024/0108758 A1
(43) Pub. Date: Apr. 4, 2024

(54) COMPOUNDS, COMPOSITIONS, AND METHODS FOR USING HLA-F

(71) Applicant: THE RESEARCH INSTITUTE AT NATIONWIDE CHILDREN'S HOSPITAL, Columbus, OH (US)

(72) Inventor: Brian KASPAR, Columbus, OH (US)

(21) Appl. No.: 18/483,090

(22) Filed: Oct. 9, 2023

Related U.S. Application Data
(63) Continuation of application No. 17/977,385, filed on Oct. 31, 2022, now abandoned.
Application No. 17/977,385 is a continuation of application No. 17/703,643, filed on Mar. 24, 2022, now abandoned.
Application No. 17/703,643 is a continuation of application No. 17/379,565, filed on Jul. 19, 2021, now abandoned.
Application No. 17/379,565 is a continuation of application No. 16/950,490, filed on Nov. 17, 2020, now abandoned.
Application No. 16/950,490 is a continuation of application No. 16/804,291, filed on Feb. 28, 2020, now abandoned.
Application No. 16/804,291 is a continuation of application No. 16/454,791, filed on Jun. 27, 2019, now abandoned.
Application No. 16/454,791 is a continuation of application No. 15/546,179, filed on Jul. 25, 2017, now abandoned.
(60) Provisional application No. 62/247,956, filed on Oct. 29, 2015.
(60) Provisional application No. 62/107,866, filed on Jan. 26, 2015.

(51) Int. Cl. A61K 48/00; A61P 25/28

(52) U.S. Cl. CPC A61K 48/0058; A61K 48/005; A61P 25/28; C12N 2750/14143

(57) ABSTRACT

The invention relates to compositions, compounds, methods, and uses for the treatment of amyotrophic lateral sclerosis. In particular, the invention relates to compounds, compositions, methods, and uses for the treatment of amyotrophic lateral sclerosis by increasing the expression of the MHC class I molecule, HLA-F, in motor neurons of the patient.

### RELATED APPLICATIONS

This application is a continuation of U.S. patent application Ser. No. 17/977,385, filed Oct. 31, 2022, which is a continuation application of U.S. patent application Ser. No. 17/703,643, filed Mar. 24, 2022, which is a continuation application of U.S. patent application Ser. No. 17/379,565, filed Jul. 19, 2021, which is a continuation of U.S. patent application Ser. No. 16/950,490, filed Nov. 17, 2020, which is a continuation application of U.S. patent application Ser. No. 16/804,291, filed Feb. 28, 2020, which is a continuation application of U.S. patent application Ser. No. 16/454,791, filed Jun. 27, 2019, which is a continuation application of U.S. patent application Ser. No. 15/546,179, filed Jul. 25, 2017, which is a national stage entry under 35 U.S.C. § 371(b) of PCT International Application No. PCT/US2016/014121, filed Jan. 20, 2016, which claims the benefit of U.S. Provisional Application No. 62/247,956 filed Oct. 29, 2015, and U.S. Provisional Application No. 62/107,866 filed Jan. 26, 2015, the entire disclosures of which are incorporated herein by reference.

### GOVERNMENT RIGHTS

This invention was made with government support under NS058224, NS064492, NS077984, and NS069476 awarded by the National Institutes of Health. The government has certain rights in the invention.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 19 kilobytes xml file named “396036.xml,” created on Dec. 12, 2023.

### TECHNICAL FIELD

The invention relates to compounds, compositions, methods, and uses for the treatment of neurodegenerative diseases (e.g., amyotrophic lateral sclerosis). In particular, the invention relates to compounds, compositions, methods, and uses for the treatment of amyotrophic lateral sclerosis by increasing the expression of the HLA-F MHC class I molecule in motor neurons of the patient.

### BACKGROUND AND SUMMARY

Amyotrophic lateral sclerosis, commonly referred to as Lou Gehrig's disease, is characterized by selective, premature degeneration and death of motor neurons in the motor cortex, brain stem and spinal cord. The loss of motor neurons causes progressive muscle paralysis ultimately leading to death from respiratory failure. Approximately 90% of all amyotrophic lateral sclerosis cases are sporadic amyotrophic lateral sclerosis, without a family history of the disease, and the other approximately 10% of cases are cases of familial amyotrophic lateral sclerosis. Despite significant efforts to identify risk factors and potential susceptibility genes, the etiology of sporadic amyotrophic lateral sclerosis remains largely unknown.

Various rodent models carrying dominant mutations of the human superoxide dismutase (SOD1) that is causative in about 20% of familial amyotrophic lateral sclerosis cases, have been instrumental to model motor neuron toxicity in amyotrophic lateral sclerosis. Insight into the mechanisms underlying motor neuron toxicity is pertinent for the development of successful therapies for amyotrophic lateral sclerosis.

Accordingly, the present inventors have discovered that overexpression of the HLA-F MHC class I molecule in motor neurons is protective against amyotrophic lateral sclerosis. The compounds, compositions, methods, and uses described herein can be used to treat sporadic or familial amyotrophic lateral sclerosis. In addition, the compounds, compositions, methods, and uses described herein may be useful for treating other neurodegenerative diseases in which neurons are lost, including but not limited to Alzheimer's disease (AD), Parkinson's disease (PD), and Huntington's disease (HD).

In one embodiment, a method for treating amyotrophic lateral sclerosis by increasing HLA-F expression in motor neurons of a patient is provided. The method comprises the step of administering to the patient a composition comprising an effective amount of a compound that increases the expression of HLA-F in the motor neurons of the patient.

In another illustrative aspect, a pharmaceutical composition is provided. The pharmaceutical composition comprises a dosage form of a compound effective to increase the expression of HLA-F in the motor neurons of a patient with amyotrophic lateral sclerosis.

In yet another aspect, a compound is provided. The compound comprises a vector operably linked to a nucleic acid comprising SEQ ID NO: 1 and a promoter for expression of the nucleic acid in a human patient.

Several embodiments of the invention are also described by the following enumerated clauses:
1. A method for treating amyotrophic lateral sclerosis by increasing HLA-F expression in motor neurons of a patient, the method comprising the step of
   administering to the patient a composition comprising an effective amount of a compound that increases the expression of HLA-F in the motor neurons of the patient.
2. The method of clause 1 wherein the increased expression of HLA-F results in an effect on motor neurons in the patient selected from the group consisting of an increase in the number of motor neurons, a decrease in soma atrophy, and an increase in neurite length after administration of the compound.
3. The method of any one of clauses 1 to 2 wherein the compound is selected from the group consisting of a drug, a peptide, and a nucleic acid.
4. The method of clause 3 wherein the compound is a nucleic acid.
5. The method of clause 4 wherein the nucleic acid comprises a bacterial vector or in a viral vector.
6. The method of clause 5 wherein the vector is a viral vector.
7. The method of clause 6 wherein the viral vector is selected from the group consisting of a lentiviral vector, an adeno-associated virus vector, and an adenovirus vector.
8. The method of any one of clauses 4 to 7 wherein the nucleic acid comprises the sequence of SEQ ID NO: 1.
9. The method of any one of clauses 4 to 7 wherein the nucleic acid comprises the sequence of SEQ ID NO: 2.
10. The method of any one of clauses 1 to 9 wherein the amyotrophic lateral sclerosis is sporadic amyotrophic lateral sclerosis.
11. The method of any one of clauses 1 to 9 wherein the amyotrophic lateral sclerosis is familial amyotrophic lateral sclerosis.
12. The method of any one of clauses 1 to 11 wherein the amount of the compound is in the range of about 1 ng/kg of patient body weight to about 1 mg/kg of patient body weight.
13. The method of any one of clauses 1 to 12 wherein the amount of the compound is in the range of about 1 ng/kg of patient body weight to about 500 ng/kg of patient body weight.
14. The method of any one of clauses 1 to 13 wherein the amount of the compound is in the range of about 1 ng/kg of patient body weight to about 100 ng/kg of patient body weight.
15. The method of any one of clauses 1 to 14 wherein the composition further comprises a carrier, an excipient, or a diluent, or a combination thereof.
16. The method of clause 15 wherein the composition comprises a pharmaceutically acceptable carrier, wherein the pharmaceutically acceptable carrier is a liquid carrier.
17. The method of clause 16 wherein the liquid carrier is selected from the group consisting of saline, glucose, alcohols, glycols, esters, amides, and a combination thereof.
18. The method of any one of clauses 1 to 17 wherein the composition is administered in a single-dose or a multiple-dose regimen.
19. The method of any one of clauses 1 to 18 wherein the compound is administered by a routes selected from the group consisting of intravenous, intrathecal, epidural, intracerebroventricular, intracranial, and subcutaneous delivery.
20. The method of any one of clauses 1 to 18 wherein the compound is administered by lumbar puncture or cisterna magna administration.
21. The method of any one of clauses 1 to 18 wherein the compound is delivered to the brain, the spinal cord, the central nervous system, or the peripheral nervous system of the patient upon administration.
22. The method of any one of clauses 1 to 18 wherein the compound is delivered to an upper or lower motor neuron of the patient upon administration.
23. A pharmaceutical composition comprising a dosage form of a compound effective to increase the expression of HLA-F in the motor neurons of a patient with amyotrophic lateral sclerosis.
24. The composition of clause 23 wherein the compound is selected from the group consisting of a drug, a peptide, and a nucleic acid.
25. The composition of clause 24 wherein the compound is a nucleic acid.
26. The composition of clause 25 wherein the nucleic acid comprises the sequence of SEQ ID NO: 1.
27. The composition of clause 25 wherein the nucleic acid comprises the sequence of SEQ ID NO: 2.
28. The composition of any one of clauses 23 to 27 wherein the composition further comprises one or more carriers, diluents, or excipients, or a combination thereof.
29. The composition of clause 28 wherein the composition comprises a pharmaceutically acceptable carrier, wherein the pharmaceutically acceptable carrier is a liquid carrier.
30. The composition of clause 29 wherein the liquid carrier is selected from the group consisting of saline, glucose, alcohols, glycols, esters, amides, and a combination thereof.
31. The composition of any one of clauses 23 to 30 wherein the purity of the compound is at least 98% based on weight percent.
32. The composition of any one of clauses 23 to 31 wherein the composition is in an ampoule or a sealed vial.
33. The composition of any one of clauses 23 to 28 or 31 to 32 in the form of a reconstitutable lyophilizate.
34. The composition of any one of clauses 23 to 28 or 31 to 32 in the form of a lyophilizate.
35. The composition of any one of clauses 23 to 28 or 31 to 34 in the form of a solid.
36. The composition of any one of clauses 25 to 35 wherein the nucleic acid further comprises a viral vector.
37. The composition of clause 36 wherein the vector is a lentiviral vector.
38. The composition of clause 36 wherein the vector is an adeno-associated virus vector.
39. Use of the composition of any one of clauses 23 to 38 for the manufacture of a medicament for treating amyotrophic lateral sclerosis.
40. The pharmaceutical composition of any one of clauses 23 to 38 for use in treating amyotrophic lateral sclerosis.
41. The method or pharmaceutical composition of any one of clauses 1 to 18 or 21 to 40 wherein the composition is in a dosage form selected from the group consisting of an inhalation dosage form, an oral dosage form, and a parenteral dosage form.
42. A compound comprising a vector operably linked to a nucleic acid comprising SEQ ID NO: 1 and a promoter for expression of the nucleic acid in a human patient.
43. The compound of clause 42 wherein the vector is a viral vector.
44. The compound of clause 43 wherein the viral vector is a lentiviral vector.
45. The compound of clause 43 wherein the vector is an adeno-associated virus vector.
46. The compound of clause 44 wherein the vector linked to the nucleic acid comprises SEQ ID NO: 2.
47. The compound of any one of clauses 42-46 wherein the promoter is a heterologous promoter.
48. A method for treating amyotrophic lateral sclerosis in a patient, the method comprising:
administering to the patient a composition comprising an effective amount of an inhibitor of an ER stressor.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIGS. 1A-1C. Spinal cord MNs express MHCI transcripts. FIG. 1A Representative images of in situ hybridization data for mouse MHCI (H2-D^{b}) along with β2m obtained from the lumbar spinal cord of a 56-day old wild-type mouse. Images were obtained by searching the interactive database of gene expression from the Allen Spinal Cord Atlas (Seattle, WA), available on http://mousespinal.brain-map.org. FIG. 1B H2-D^{b} transcripts were also detected by us in the MNs of the ventral horn in the lumbar spinal cord of a 60-day old wild-type mouse but not in an age matched H2-K^{b} and H2-D^{b}-deficient mouse. In dark-field micrographs, in situ hybridized riboprobes appeared white. Scale bars, 500 μm (FIG. 1A), 400 μm (FIG. 1B), 100 μm (FIG. 1A inset). FIG. 1C Representative images of in situ hybridization data for mouse MHCI (H2-Ke2, H2-D1) along with β2m obtained from the lumbar spinal cord of a 56-day old wild-type mouse. Images were obtained by searching the interactive database of gene expression from the Allen Spinal Cord Atlas.

FIGS. 2A-2D. At end-stage of ALS spinal cords, MNs display marked reduction in MHCI expression. FIG. 2A Representative images showing marked reduction of MHCI (H2-D^{b}/H2-K^{b}) expression in MNs at late stage of disease in the SOD1^{G93A} ALS animal model by immunofluorescence analysis. FIG. 2B Percent of MHCI positive lumbar spinal cord MNs found in SOD1^{G93A} and control mice evaluated as shown in FIG. 2A. For each group, two animals were used to obtain spinal cord sections. 321, 216, 216, 154 MNs were counted in graph columns 1 through 4. FIG. 2C DAB immunohistochemistry analysis revealed marked reduction of MHCI (HLA-ABC) expression in MNs of post-mortem ALS patient's spinal cord. Arrowheads point to MNs. FIG. 2D Percent of MHCI positive MNs found in human spinal cords of ALS patients and controls determined as shown in FIG. 2C. 50, 60, 35, 51, 71, 87, 45, 46, 68 and 22 of MNs were counted in graph columns 1 through 10. WT, wild-type. SOD1, SOD1^{G93A}. Scale bars 20 μm. MHC+=green and MHC−=red.

FIGS. 3A & 3B. Reduction of MHCI expression in MNs also occurs in cervical and thoracic segments of the spinal cords in SOD1^{G93A} mice at age P125. Representative images showing marked reduction of MHCI (H2-K^{b}D^{b}) expression in MNs in both cervical (FIG. 3A) and thoracic FIG. 3B spinal cords at late stage of disease in the SOD1^{G93A} ALS mouse model by immunofluorescence analysis. Percent of MHCI positive spinal MNs found in SOD1^{G93A} and control mice were quantified. For each group, spinal cord sections were obtained from two animals and a total of 199, 277, 208, 212 of MNs in FIG. 3A and a total of 99, 110, 142, 110 of MNs in FIG. 3B were counted corresponding to graph columns 1-4, respectively. WT, wild-type. SOD1, SOD1^{G93A}. Scale bars 20 μm. MHC+=green and MHC−=red.

FIGS. 4A-4C. iPS cell derived MNs share gene expression profile with MNs derived from ES cells. FIG. 4A Schematic representation of the process used to generate MNs from Hb9::GFP iPS and ES cells. MN cultures were purified by FACS prior to experimental use. FIGS. 4B and 4C SOD1^{WT} and SOD1^{G93A} iPS cell derived MNs were morphologically similar to MNs derived from ES cells (FIG. 4B) and expressed similar levels of the prototypic MNs markers that were analyzed (FIG. 4C). Data show the mean±s.e.m fold induction of triplicates. Black=mESC MNs, green=WT iPS MNs, and red=G93A iPS MNs.

FIGS. 5A-5C. ALS astrocytes induce down-regulation of MHCI expression in MNs. FIG. 5A Wild-type and SOD1^{G93A} MNs displayed similar levels of MHCI. FIG. 5B Co-culture of MNs with SOD1 mice derived microglia do not result in MHCI expression changes in MN compared with wild type mice derived microglia. Images shown were collected at 72 h. FIG. 5C Time dependent MNs MHCI down-regulation upon co-culture with SOD1 astrocytes. Data in FIGS. 5A-5C is a representative experiment out of four independent experiments each with three replicates. Levels of MHCI are expressed as mean fluorescence intensity (MFI) found in MNs. Error bars represent s.e.m. MHCI levels shown in graphs of FIGS. 5A-5C are displayed as relative to WT, 24 hours. Each dot in the graphs represents MHCI level found per MN (One-Way ANOVA, *P<0.05; ***P<0.001; ns, non-significant P≥0.5). WT, wild-type. SOD1, SOD1^{G93A}. Scale bars 10 μm.

FIGS. 6A-6G. NPC derived astrocytes express prototypic astrocytic markers and are devoid of other glia types. FIG. 6A Immunofluorescence marker analysis revealed that both wild-type and SOD1^{G93A} NPC derived astrocytes expressed high levels of astrocytic markers, GFAP, S100β, the glutamate transporter, EAAT2. Expression of the microglia marker Iba1 and oligodendrocyte marker MBP were found absent. FIGS. 6B and 6C Quantitative RT-PCR analysis of wild-type and SOD1^{G93A} NPC derived astrocytes detects very little to no expression of the microglia markers, Iba1 and Cd11b, and the oligodendrocyte markers Mbp and Plp1. WT, wild-type. SOD1, SOD1^{G93A}. Scale bar, 1 mm. Mouse and human NPC derived astrocytes express prototypic astrocytic markers and are devoid of CTL contaminants. FIG. 6D RNA analysis showed that mouse NPCs (WT and SOD1^{G93A}) used as a source for astrocytes, were free from NK and CTL cell contamination. FIG. 6E Immunofluorescence marker analysis revealed that both WT and SOD1^{G93A} NPC derived astrocytes expressed high levels of prototypic astrocytic markers, GFAP and S100β, as well as the glutamate transporter, EAAT2. NPC derived astrocytes did not express the CTL marker CD8A. FIGS. 6F and 6G RNA analysis for the prototypic CTL markers failed to detect CD3E and CD8A in NPC derived astrocytes from both WT and SOD1^{G93A} mice FIG. 6F and in human astrocytes FIG. 6G. WT, wild-type. SOD1, SOD1^{G93A}. Scale bar, 1 mm.

FIGS. 7A-7D. MHCI is not down-regulated in GABAergic neurons in the presence of SOD1^{G93A} astrocytes. FIG. 7A In contrast to MNs, no overt change in the levels of MHCI (H2-K^{b}D^{b}) expression was observed in GABAergic neurons upon co-culture with SOD1^{G93A} astrocytes versus wild-type astrocytes. Data represent one of three independent experiments run in triplicate and shown as mean±s.e.m of MHCI fluorescence intensity in GAD67⁺ cells. MHCI levels shown in graph are displayed as relative to WT, 24 hours. Each dot in the graphs represents MHCI level found per GAD67⁺ cell. (One-Way ANOVA, ns, non-significant P≥0.5). WT, wild-type astrocytes. SOD1, SOD1^{G93A} astrocytes. Scale bars 5 μm. Knockdown of H2-K^{b} expression in GABAergic neurons results in susceptibility to SOD1^{G93A} astrocyte toxicity. FIGS. 7B and 7C Induction of H2-K^{b} shRNA resulted in efficient down regulation of H2-K^{b} expression in GABAergic neurons as shown by RNA (b, upper panel), protein expression as shown by fluorescence imaging FIG. 7B, lower panel), and MHCI quantification FIG. 7C. Data in FIG. 7A and FIG. 7C represent one of three independent experiments run in triplicate and shown as mean±s.e.m of MHCI fluorescence intensity in GAD67+ cells. Each dot in the graphs represents MHCI level found per GAD67+ cell. (One-Way ANOVA, ***P<0.001; ns, non-significant P≥0.5). FIG. 7D Upon co-culture with SOD1^{G93A} astrocytes, unlike scrambled shRNA treated GABAergic neurons, GABAergic neurons treated with H2-K^{b} shRNA showed a decrease in cell survival FIG. 7D; measurements were taken at 120 h of co-culture with astrocytes). Representative data of three independent experiments and shown by the mean±s.e.m of counts found in 3 wells per group (One-Way ANOVA, ***P<0.001; ns, non-significant P≥0.5). WT, wild-type. SOD1, SOD1^{G93A}. Ast, astrocytes. Scale bars 5 μM.

FIGS. 8A-8D. Lentiviral transduction of MNs with H2-K^{b} allows sustained MHCI expression in MNs despite co-culture with SOD1^{G93A} astrocytes. FIG. 8A Transduction of 293 cells with lentivirus particles encoding H2-K^{b}, here referred to as Lv-H2K showed expression of H2-K^{b} transgene, as detected by RT-PCR. FIG. 8B Infection of Hb9::GFP MNs with lentivirus allowed a high level of transduction. RFP encoded by the lentivirus was visualized 72 hours post-infection. FIG. 8C Upon co-culture with SOD1^{G93A} astrocytes, MHCI staining showed reduced expression of MHCI on MNs transduced with Lv-RFP but sustained expression of MHCI on MN transduced with Lv-H2K. FIG. 8D Quantification of MHCI levels as shown in FIG. 8C. Data represents one of three experiments performed independently with triplicates and shows mean±s.e.m of MHCI fluorescence intensity in MNs. MHCI levels shown in d are displayed as relative to WT. Each dot in the graphs represents MHCI level found per MN cell. (One-Way ANOVA, ***P<0.001; ns, non-significant P≥0.5). MHCI expression was determined 72 hours post initiation of co-culture (c and d). WT, wild-type. SOD1, SOD1^{G93A}. Ast, astrocytes. Scale bars, 200 μm FIG. 8B, 20 μm FIG. 8C.

FIG. 9A-9G. H2-K^{b} overexpression protects MNs from ALS astrocytes induced toxicity and delays disease progression in SOD1^{G93A} mouse model. FIGS. 9A and 9B Overexpression of H2-K^{b} (a mouse MHCI isoform) but not H2-D^{b} or H2-L^{d} in mouse MNs protected them from SOD1^{G93A} astrocyte toxicity FIG. 9A as shown by increase in Hb9::GFP+ MN counts FIG. 9B. Data shown is a representative of three independent experiments and is displayed as the mean±s.e.m of counts found in 3 wells (One-Way ANOVA, ****P<0.0001; ns, non-significant P≥0.5). Scale bar 100 μm. FIG. 9C Kaplan-Meier survival curve of SOD1^{G93A} mice injected at day 1 with AAV9-H2K (n=28; top line), AAV9-H2D (n=14; middle line) or AAV9-empty controls (n=26; bottom line). Mean survival was 156.9±2.6 days in AAV9-H2K, 139.2±1.4 days in AAV9-H2D and 135.5±1.6 days in controls (unpaired t-test, mean±s.e.m, P<0.0001). FIGS. 9D and 9E AAV9-H2K injected SOD1^{G93A} mice (line extending to 120 days in graph (FIG. 9G) reached mean onset at 103.3±2.0 days, 103.1±1.2 days in AAV9-H2D and 99.73±1.2 days in controls (unpaired t-test, mean±s.e.m, P=0.1). FIG. 9F Mean disease progression observed in AAV9-H2K injected SOD1^{G93A} mice was 52.7±2.6 days, 34.62±2.2 days in AAV9-H2D and 34.1±1.8 days in controls (unpaired t-test, mean±s.e.m, P<0.0001). FIG. 9G Rotarod performance of AAV9-H2K treated SOD1^{G93A} mice (top line) compared with age-matched controls (n equals 10 to 28 with lower numbers observed closed to end-stage, unpaired t-test, mean±s.e.m, *P<0.05; **P<0.01; ***P<0.005). WT, wild-type. SOD1, SOD1^{G93A}. Ast, astrocytes.

FIGS. 10A & 10B. CNS delivery of AAV9 at birth results in efficient spinal MN transduction. FIG. 10A High levels of spinal cord MNs were transduced when AAV9-GFP was delivered via cerebral spinal fluid at postnatal day 1. ChAT was used as a marker to highlight MNs. FIG. 10B Increased expression of H2-K^{b} or H2-D^{b} in spinal cord was observed 50 days after CNS delivery of AAV9-H2K. Scale bar 50 μm.

FIGS. 11A & 11B. H2-K^{b} expression in SOD1^{G93A} astrocytes does not protect MNs from SOD1^{G93A} astrocyte toxicity. FIG. 11A Astrocytes were readily transduced with lentivirus as shown here by the expression of RFP. FIG. 11B H2-K^{b} overexpression in SOD1^{G93A} astrocytes did not protect MNs from SOD1^{G93A} astrocyte toxicity as shown by no difference in the number of Hb9::GFP+ MN counts observed when SOD1^{G93A} astrocytes were infected either with Lv-RFP or Lv-H2K::RFP. Data represents one of three independent experiments and is shown as the mean±s.e.m of counts found in 3 wells per experimental group. (One-Way ANOVA, ****P<0.0001). WT, wild-type. SOD1, SOD1^{G93A}. Scale bar 200 μm.

FIGS. 12A-12J. ALS astrocytes express MHCI inhibitory receptors. FIGS. 12A and 12B Expression of MHCI inhibitory receptors LY49C and LY49I occurred in the spinal cord of SOD1^{G93A} mice at disease end-stage as determined by RNA (FIG. 12A) and immunohistochemistry analysis (FIGS. 12B and 12I). FIGS. 12E, 12J, 12D and 12E SOD1^{G93A} mouse astrocytes expressed high levels of Ly49C and LY49I as shown in vivo immunohistochemistry analysis (FIGS. 12C and 12J) and in vitro by RNA (FIG. 12D) and immunohistochemistry analysis FIG. 12E). FIGS. 12F-12H Expression of the MHCI inhibitory receptor KIR3DL2 occurred specifically in human ALS astrocytes as determined by RNA analysis of in vitro cultured astrocytes (FIG. 12F) and in sections of spinal cord of a representative SALS post-mortem tissue FIGS. 12G and 12H. WT, wild-type. SOD1, SOD1^{G93A}. Scale bars 50 μm (FIG. 12B and FIG. 12I), 200 μm (FIGS. 12E and 12G), 10 μm (FIGS. 12E and 12J), 5 μm (FIG. 12H).

FIG. 13. MHCI inhibitory receptors are expressed in SOD1^{G93A} astrocytes and cytotoxic T lymphocytes. At disease end-stage of SOD1^{G93A} mice, LY49C and LY49I expression were observed in CD8A positive CTLs infiltrated in spinal cord, but also in the majority of astrocytes. LY49C/I positive CTLs and astrocytes were not found in spinal cords of wild-type littermates. Arrow indicates LY49C/I positive astrocytes. Arrowhead indicates LY49C/I positive CTL. WT, wild-type. SOD1, SOD1^{G93A}. Scale bar 20 μm.

FIGS. 14A-14F. HLA-F expression in human MNs protects them from ALS astrocytes induced toxicity. FIG. 14A DAB immunohistochemical analysis revealed marked reduction of HLA-F expression in MNs of post-mortem ALS patient's spinal cord. Green arrowheads point to MNs. FIG. 14B Percent of HLA-F positive MNs found in human spinal cords of ALS patients and control determined as shown in FIG. 14A. For columns 1 through 5, the total number of MNs was 62, 58, 54, 26 and 42, respectively (scale bars 20 μm). FIG. 14C Human ESC derived MNs showed morphological neuronal features and expressed high levels of prototypic MN markers. FIG. 14D Lentivirus infected cells expressed both transgenes, human HLA-F and eGFP as shown by RNA (upper panel) and immunocytochemistry analysis (lower panel). FIGS. 14E and 14F HLA-F expression in human MNs protected them from FALS and SALS astrocyte induced toxicity as visualized by representative images of ChAT positive MNs (FIG. 14E) and by the quantification of number of ChAT positive cells FIG. 14F upon co-culture with astrocytes. Dotted line represents average MN counts when co-cultured with non-ALS controls. Data shows a representative of three independent experiments and is displayed as the mean±s.e.m counts of triplicates. (One Way ANOVA, *P<0.05; **P<0.01; ***P<0.001; ns, nonsignificant P≥0.5). Scale bars 20 μm (FIG. 14A), 50 μm (FIG. 14C), 100 μm (FIG. 14E).

FIG. 15. At the symptomatic stage SOD1 mice show increased expression of MHCI in the sciatic nerve axons fibers. Scale bar 20 μm.

FIGS. 16A-16H. Mouse and human NPC derived astrocytes are devoid of CTL and NK contaminants. RNA analysis showed that mouse (FIGS. 16A-16D) and human (FIGS. 16E-16H) NPCs derived astrocytes used in this study were free from CTL (FIGS. 16A and 16B and FIGS. 16E and 16F) and NK (FIGS. 16C and 16D and FIGS. 16G and 16H and) cell contaminants. SC, Spleenocytes.

FIGS. 17A-17B. SOD1^{G93A} mutation in MNs induces down-regulation of MHCI but this reduction is not further increased by the presence of SOD1^{G93A} astrocytes. FIG. 17A When cultured alone, MNs expressing SOD1^{G93A} mutation retain MHCI expression for the first 72 hours but show a 27% down-regulation of MHCI compared to WT MNs by 120 hours. Images shown were collected at 72 h. FIG. 17B In the presence of SOD1^{G93A} astrocytes, SOD1^{G93A} MNs showed reduced but similar levels of MHCI expression as WT MNs cultured with SOD1^{G93A} astrocytes. Data in (FIGS. 17A and 17B) are representative of three independent experiments performed in triplicates. Data are shown as mean±s.e.m of MHCI fluorescence intensity found in MNs were displayed relative to WT MNs cultured in the absence of astrocytes. Each dot in the graphs represents MHCI level found per MN (One-Way ANOVA, *P<0.05; ns, non-significant P≥0.5). WT, iPSC derived MNs expressing normal SOD1. SOD1, iPSC derived MNs expressing SOD1^{G93A} mutation. Scale bars 10 μm.

FIGS. 18A and 18B. Down-regulation of MHCI in MNs is observed in the presence of SOD1^{G93A} astrocyte conditioned medium and an endoplasmic reticulum (ER) stressor. FIG. 18A Culturing of MNs with SOD1^{G93A} astrocyte conditioned medium led to a specific and marked down-regulation of MHCI (H2-K^{b}D^{b}) expression. FIG. 18B Among a subset of molecules known to be secreted by SOD1^{G93A} astrocytes and to cause MN stress, only the ER stressor, thapsigargin, markedly down-regulated MHCI in MNs. Data shown were collected 24 hours post astrocyte conditioned medium incubation in FIG. 18A and 9 h post drugs administration in FIG. 18B. Data shown are representative of three independent experiments with triplicates and shown as mean±s.e.m of MHCI fluorescence intensity found in MNs. MHCI levels shown in graphs a and b are displayed as relative to each control. Each dot in the graphs represents MHCI level found per MN (One-Way ANOVA, *P<0.05; ****P<0.0001; ns, non-significant P≥0.5). WT, wild-type. SOD1, SOD1^{G93A}. CM, conditioned medium. Scale bars 10 μm.

FIGS. 19A-19C. H2-K^{b} knockdown in MNs does not alter their viability in culture or susceptibility to known ALS stress molecules. H2-K^{b} knockdown in MNs did not alter MN cell viability during the culture period FIG. 19A or increased susceptibility to the ER stressor molecule thapsigargin FIG. 19B or increased susceptibility to reactive oxygen species generating molecule menadione FIG. 19C. scr, scrambled.

FIG. 20. H2-K^{b} knockdown in MNs increases susceptibility to SOD1^{G93A} astrocytes toxicity. MNs treated with a lentivirus expressing H2-K^{b} shRNA did not show a difference in survival compared to scrambled shRNA control in the presence of wild-type astrocytes, but showed a decrease in cell survival throughout the culture period in the presence of SOD1^{G93A} astrocytes. Statistical analysis was performed for the comparison between red and purple (Two-Way ANOVA, *P<0.05; **P<0.01). scr, scrambled.

FIG. 21A-21B. Knockdown of KIR3DL2 in ALS astrocytes leads to an increase in the rate and toxicity level of ALS astrocytes. FIG. 21A RT-PCR analysis show that the Kir3dl2 shRNA used in this study is effective at knocking down KIR3DL2 expression in ALS astrocytes. FIG. 21B Levels of MN death observed at both 7 and 14 days post co-culture with astrocytes. ALS astrocytes were infected with either scrambled shRNA or Kir3dl2 shRNA prior to co-culture. KIR3DL2 suppression in ALS astrocytes resulted in a faster and an increased MN death. FALS, familial ALS. SALS, sporadic ALS. scr, scrambled.

FIG. 22. Robust HLA-F expression is observed in human ES-derived MN cells upon transduction with LV-HLA-F:GFP. Human ES-derived MN progenitor cells express HLA-F at low levels (red) upon differentiation (no Lv, upper panel). Upon transduction with Lv-HLA-F:GFP, transduced cells identified by green fluorescent protein expression (GFP; green), display high levels of HLA-F expression (red, Lower panel). Scale bar 20 μm.

### DETAILED DESCRIPTION OF ILLUSTRATIVE EMBODIMENTS

Several embodiments of the invention are described in this Detailed Description section of the patent application and each of the embodiments described in this Detailed Description section of the application applies to each of the embodiments, or combinations thereof, described in the Background and Summary section of the patent application.

In any of the various embodiments described herein, the following features may be present where applicable, providing additional embodiments of the invention. For all of the embodiments, any applicable combination of embodiments is also contemplated.

The methods, uses, compounds, and compositions described herein can be used to treat either sporadic or familial amyotrophic lateral sclerosis, and can be used for both human clinical medicine and veterinary medicine. In addition, the methods, uses, compounds, and compositions described herein may be useful for treating other neurodegenerative diseases in which neurons are lost, including but not limited to AD, PD, and HD. In one aspect, the patient can have a mutation in SOD1. In one embodiment, the compounds described herein that can be used to treat sporadic or familial amyotrophic lateral sclerosis are compounds that are effective to increase the expression of the MHC class I molecule, HLA-F, in the motor neurons of a patient with amyotrophic lateral sclerosis. The compounds are selected from the group consisting of drugs, peptides, and nucleic acids, or combinations thereof. In some embodiments, the compositions described herein that can be used to treat amyotrophic lateral sclerosis include an inhibitor of an ER stressor. Representative inhibitors of ER stressors that may be used include but are not limited to inducers of expression and activity of chaperones (e.g., lithium, valproate, BIX), inhibitors of PERK-eIF2-alpha phosphatase (e.g., salubrinal, guanabenz), inducers of antioxidant pathways (e.g., carnosic acid, triterpenoids), stress kinase inhibitors (e.g., JNK inhibitors, P38 inhibitors), antioxidants (e.g., kaempferol, beicalein, apigenin), chemical chaperones (e.g., tauroursodeoxycholic acid or TUDCA, sodium 4-phenylbutyrate or 4-PBA), and the like (see: Kim, I., et al. “Cell death and endoplasmic reticulum stress: disease relevance and therapeutic opportunities,” Nature Reviews Drug Discovery 7, 1013-1030 (2008); Kraskiewicz, H., et al. “InterfERing with endoplasmic reticulum stress,” Trends Pharmacol Sci. 33:53-63 (2012); and Schönthal, A. H. “Endoplasmic reticulum stress: its role in disease and novel prospects for therapy,” Scientifca, 857516 (2012)).

In the embodiment where the compounds are nucleic acids, suitable methods for delivery of the nucleic acids, such as full-length coding sequences, antisense RNA molecules, siRNAs, shRNAs, or miRNAs to a patient with amyotrophic lateral sclerosis include bacterial or viral vectors, such as lentiviral vectors, adeno-associated virus vectors, or adenovirus vectors. Exemplary of such nucleic acids are the nucleic acids with SEQ ID NO: 1 and SEQ ID NO: 2 (see Table 1).

In another embodiment, the compounds can be drugs such as interferones, LPS, Ganoderma lucidum polysaccharides, topotecan, trichostatin A, polylactic-co-glycolic acid nanoparticles, or mesoporous silicon microparticles.

For embodiments in which the compound includes a vector operably linked to a nucleic acid and a promoter for expression of the nucleic acid in a human patient, the promoter may, in some embodiments, be a heterologous promoter. Representative heterologous promoters that may be used to control the expression of HLA-F in neuronal cells include but are not limited to human or synthetic promoters, including but not limited to neuron-specific enolase (NSE), Hb9, choline acetyltransferase (ChAT), synapsin, CMV early enhancer/chicken beta actin (CAG) promoter, cytomegalovirus promoter (CMV), and the like.

In the embodiment where the method of delivery is a viral vector, the viral vector can be operatively linked to a full-length coding sequence, or to an siRNA, shRNA, or miRNA (e.g., by a promoter that is functional in the target cells such as cells of a human patient). In one embodiment, the viral vector is single-stranded. In one illustrative aspect, the viral vector can be an adeno-associated viral vector, for example, AAV serotype AAV-1, AAV-2, AAV-3, AAV-4, AAV-5, AAV-6, AAV-7, AAV-8, AAV-9, AAV-10, AAV-11, or AAVrh74. The sequences of the genomes of these AAV serotypes are known in the art. Techniques for producing AAV are known in the art and are described in WO 01/83692, U.S. 20050053922 and U.S. 20090202490, each of which is incorporated herein by reference.

In the embodiment where the compounds described herein are compounds that are effective to increase the expression of the MHC class I molecule, HLA-F, in the motor neurons of a patient with amyotrophic lateral sclerosis, the compounds can be selected from the group consisting of drugs, peptides, and nucleic acids, or combinations thereof. In an illustrative embodiment, the nucleic acid with SEQ ID NO: 1 or SEQ ID NO: 2, encoding the histocompatibility complex HLA-F, shown herein to cause sustained expression of MHC class I molecules in motor neurons, protecting motor neurons from the toxic effects of human ALS astrocytes, can be used to treat amyotrophic lateral sclerosis.

In accordance with these embodiments, compounds or compositions are provided comprising a purified nucleic acid comprising, or consisting of, a sequence of SEQ ID NO: 1 or SEQ ID NO: 2 (see Table 1). In this embodiment, SEQ ID NO: 1 is the HLA-F coding sequence and SEQ ID NO: 2 is the HLA-F coding sequence along with the sequence of a lentiviral vector. A purified nucleic acid is also provided comprising a complement of SEQ ID NO: 1 or SEQ ID NO: 2, or a sequence that hybridizes under highly stringent conditions to a complement of a sequence consisting of SEQ ID NO: 1 or SEQ ID NO: 2. In accordance with the invention “highly stringent conditions” means hybridization at 65° C. in 5×SSPE and 50% formamide, and washing at 65° C. in 0.5×SSPE. Conditions for high, low, and moderately stringent hybridization are described in Sambrook et al., “Molecular Cloning: A Laboratory Manual”, 3rd Edition, Cold Spring Harbor Laboratory Press, (2001), incorporated herein by reference. In some illustrative aspects, hybridization occurs along the full-length of the nucleic acid.

TABLE 1
SEQ ID NO: 1
ATGGCGCCCCGAAGCCTCCTCCTGCTGCTCTCAGGGGCCCTGGCCCTGAC
CGATACTTGGGCAGGCTCCCACTCCTTGAGGTATTTCAGCACCGCTGTGT
CGCGGCCCGGCCGCGGGGAGCCCCGCTACATCGCCGTGGAGTACGTAGAC
GACACGCAATTCCTGCGGTTCGACAGCGACGCCGCGATTCCGAGGATGGA
GCCGCGGGAGCCGTGGGTGGAGCAAGAGGGGCCGCAGTATTGGGAGTGGA
CCACAGGGTACGCCAAGGCCAACGCACAGACTGACCGAGTGGCCCTGAGG
AACCTGCTCCGCCGCTACAACCAGAGCGAGGCTGGGTCTCACACCCTCCA
GGGAATGAATGGCTGCGACATGGGGCCCGACGGACGCCTCCTCCGCGGGT
ATCACCAGCACGCGTACGACGGCAAGGATTACATCTCCCTGAACGAGGAC
CTGCGCTCCTGGACCGCGGCGGACACCGTGGCTCAGATCACCCAGCGCTT
CTATGAGGCAGAGGAATATGCAGAGGAGTTCAGGACCTACCTGGAGGGCG
AGTGCCTGGAGTTGCTCCGCAGATACTTGGAGAATGGGAAGGAGACGCTA
CAGCGCGCAGATCCTCCAAAGGCACACGTTGCCCACCACCCCATCTCTGA
CCATGAGGCCACCCTGAGGTGCTGGGCCCTGGGCTTCTACCCTGCGGAGA
TCACGCTGACCTGGCAGCGGGATGGGGAGGAACAGACCCAGGACACAGAG
CTTGTGGAGACCAGGCCTGCAGGGGATGGAACCTTCCAGAAGTGGGCCGC
TGTGGTGGTGCCTTCTGGAGAGGAACAGAGATACACATGCCATGTGCAGC
ACGAGGGGCTGCCCCAGCCCCTCATCCTGAGATGGGAGCAGTCTCCCCAG
CCCACCATCCCCATCGTGGGCATCGTTGCTGGCCTTGTTGTCCTTGGAGC
TGTGGTCACTGGAGCTGTGGTCGCTGCTGTGATGTGGAGGAAGAAGAGCT
CAGATAGAAACAGAGGGAGCTACTCTCAGGCTGCAGCCTACTCAGTGGTC
AGCGGACTCTTGATGATAACATGGTGGTCAAGCTTATTTCTCCTGGGGGT
GCTCTTCCAAGGATATTTGGGCTGCCTCCGGAGTCACAGTGTCTTGGGCC
GCCGGAAGGTGGGTGACATGTGGATCTTGTTTTTTTTGTGGCTGTGGACA
TCTTTCAACACTGCCTTCTTGGCCTTGCAAAGCCTTCGCTTTGGCTTCGG
CTTTAGGAGGGGCAGGAGCTTCCTTCTTCGTTCTTGGCACCATCTTATGA
AAAGGGTCCAGATTAAGATTTTTGACTAG
SEQ ID NO: 2
AGCGGCCCGCGTCTGGAACAATCAACCTCTGGATTACAAAATTTGTGAAA
GATTGACTGGTATTCTTAACTATGTTGCTCCTTTTACGCTATGTGGATAC
GCTGCTTTAATGCCTTTGTATCATGCTATTGCTTCCCGTATGGCTTTCAT
TTTCTCCTCCTTGTATAAATCCTGGTTGCTGTCTCTTTATGAGGAGTTGT
GGCCCGTTGTCAGGCAACGTGGCGTGGTGTGCACTGTGTTTGCTGACGCA
ACCCCCACTGGTTGGGGCATTGCCACCACCTGTCAGCTCCTTTCCGGGAC
TTTCGCTTTCCCCCTCCCTATTGCCACGGCGGAACTCATCGCCGCCTGCC
TTGCCCGCTGCTGGACAGGGGCTCGGCTGTTGGGCACTGACAATTCCGTG
GTGTTGTCGGGGAAGCTGACGTCCTTTCCATGGCTGCTCGCCTGTGTTGC
CACCTGGATTCTGCGCGGGACGTCCTTCTGCTACGTCCCTTCGGCCCTCA
ATCCAGCGGACCTTCCTTCCCGCGGCCTGCTGCCGGCTCTGCGGCCTCTT
CCGCGTCTTCGCCTTCGCCCTCAGACGAGTCGGATCTCCCTTTGGGCCGC
CTCCCCGCCTGGAATTAATTCTGCAGTCGAGACCTAGAAAAACATGGAGC
AATCACAAGTAGCAATACAGCAGCTACCAATGCTGATTGTGCCTGGCTAG
AAGCACAAGAGGAGGAGGAGGTGGGTTTTTCCAGTCACACCTCAGGACCT
TTAAGACCAATGACTTACAAGGCAGCTGTAGATCTTAGCCACTTTTTAAA
AGAAAAGAGGGGACTGGAAGGGCTAATTCACTCCCAACGAAGACAAGATC
TGCTTTTTGCCTGTACTGGGTCTCTCTGGTTAGACCAGATCTGAGCCTGG
GAGCTCTCTGGCTAACTAGGGAACCCACTGCTTAAGCCTCAATAAAGCTT
GCCTTGAGTGCTTCAAGTAGTGTGTGCCCGTCTGTTGTGTGACTCTGGTA
ACTAGAGATCCCTCAGACCCTTTTAGTCAGTGTGGAAAATCTCTAGCAGT
AGTAGTTCATGTCATCTTATTATTCAGTATTTATAACTTGCAAAGAAATG
AATATCAGAGAGTGAGAGGCTAGCGTTTTACCGTCGACCTCTAGCTAGAG
CTTGGCGTAATCATGGTCATAGCTGTTTCCTGTGTGAAATTGTTATCCGC
TCACAATTCCACACAACATACGAGCCGGAAGCATAAAGTGTAAAGCCTGG
GGTGCCTAATGAGTGAGCTAACTCACATTAATTGCGTTGCGCTCACTGCC
CGCTTTCCAGTCGGGAAACCTGTCGTGCCAGCTGCATTAATGAATCGGCC
AACGCGCGGGGAGAGGCGGTTTGCGTATTGGGCGCTCTTCCGCTTCCTCG
CTCACTGACTCGCTGCGCTCGGTCGTTCGGCTGCGGCGAGCGGTATCAGC
TCACTCAAAGGCGGTAATACGGTTATCCACAGAATCAGGGGATAACGCAG
GAAAGAACATGTGAGCAAAAGGCCAGCAAAAGGCCAGGAACCGTAAAAAG
GCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAA
GATACCAGGCGTTTCCCCCTGGAAGCTCCCTCGTGCGCTCTCCTGTTCCG
ACCCTGCCGCTTACCGGATACCTGTCCGCCTTTCTCCCTTCGGGAAGCGT
GGCGCTTTCTCATAGCTCACGCTGTAGGTATCTCAGTTCGGTGTAGGTCG
TTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGA
CTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTAC
ACTAGAAGAACAGTATTTGGTATCTGCGCTCTGCTGAAGCCAGTTACCTT
CGGAAAAAGAGTTGGTAGCTCTTGATCCGGCAAACAAACCACCGCTGGTA
GCGGTTTTTTTGTTTGCAAGCAGCAGATTACGCGCAGAAAAAAAGGATCT
CAAGAAGATCCTTTGATCTTTTCTACGGGGTCTGACGCTCAGTGGAACGA
AAACTCACGTTAAGGGATTTTGGTCATGAGATTATCAAAAAGGATCTTCA
CCTAGATCCTTTTAAATTAAAAATGAAGTTTTAAATCAATCTAAAGTATA
TATGAGTAAACTTGGTCTGACAGTTACCAATGCTTAATCAGTGAGGCACC
TATCTCAGCGATCTGTCTATTTCGTTCATCCATAGTTGCCTGACTCCCCG
TCGTGTAGATAACTACGATACGGGAGGGCTTACCATCTGGCCCCAGTGCT
GCAATGATACCGCGAGACCCACGCTCACCGGCTCCAGATTTATCAGCAAT
AAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGT
TCGCCAGTTAATAGTTTGCGCAACGTTGTTGCCATTGCTACAGGCATCGT
GGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGC
TCCTTCGGTCCTCCGATCGTTGTCAGAAGTAAGTTGGCCGCAGTGTTATC
ACTCATGGTTATGGCAGCACTGCATAATTCTCTTACTGTCATGCCATCCG
TAAGATGCTTTTCTGTGACTGGTGAGTACTCAACCAAGTCATTCTGAGAA
TAGTGTATGCGGCGACCGAGTTGCTCTTGCCCGGCGTCAATACGGGATAA
TACCGCGCCACATAGCAGAACTTTAAAAGTGCTCATCATTGGAAAACGTT
CTTCGGGGCGAAAACTCTCAAGGATCTTACCGCTGTTGAGATCCAGTTCG
ATGTAACCCACTCGTGCACCCAACTGATCTTCAGCATCTTTTACTTTCAC
CAGCGTTTCTGGGTGAGCAAAAACAGGAAGGCAAAATGCCGCAAAAAAGG
GAATAAGGGCGACACGGAAATGTTGAATACTCATACTCTTCCTTTTTCAA
TATTATTGAAGCATTTATCAGGGTTATTGTCTCATGAGCGGATACATATT
TGAATGTATTTAGAAAAATAAACAAATAGGGGTTCCGCGCACATTTCCCC
GAAAAGTGCCACCTGACGTCGACGGATCGGGAGATCAACTTGTTTATTGC
AGCTTATAATGGTTACAAATAAAGCAATAGCATCACAAATTTCACAAATA
AAGCATTTTTTTCACTGCATTCTAGTTGTGGTTTGTCCAAACTCATCAAT
GTATCTTATCATGTCTGGATCAACTGGATAACTCAAGCTAACCAAAATCA
TCCCAAACTTCCCACCCCATACCCTATTACCACTGCCAATTACCCTGTGG
GCGCAATTAACCCTCACTAAAGGGAACAAAAGCTGGAGCTGCAAGCTTAA
TGTAGTCTTATGCAATACTCTTGTAGTCTTGCAACATGGTAACGATGAGT
TAGCAACATGCCTTACAAGGAGAGAAAAAGCACCGTGCATGCCGATTGGT
GGAAGTAAGGTGGTACGATCGTGCCTTATTAGGAAGGCAACAGACGGGTC
TGACATGGATTGGACGAACCACTGAATTGCCGCATTGCAGAGATATTGTA
TTTAAGTGCCTAGCTCGATACATAAACGGGTCTCTCTGGTTAGACCAGAT
CTGAGCCTGGGAGCTCTCTGGCTAACTAGGGAACCCACTGCTTAAGCCTC
AATAAAGCTTGCCTTGAGTGCTTCAAGTAGTGTGTGCCCGTCTGTTGTGT
GACTCTGGTAACTAGAGATCCCTCAGACCCTTTTAGTCAGTGTGGAAAAT
CTCTAGCAGTGGCGCCCGAACAGGGACTTGAAAGCGAAAGGGAAACCAGA
GGAGCTCTCTCGACGCAGGACTCGGCTTGCTGAAGCGCGCACGGCAAGAG
GCGAGGGGCGGCGACTGGTGAGTACGCCAAAAATTTTGACTAGCGGAGGC
TAGAAGGAGAGAGATGGGTGCGAGAGCGTCAGTATTAAGCGGGGGAGAAT
TAGATCGCGATGGGAAAAAATTCGGTTAAGGCCAGGGGGAAAGAAAAAAT
ATAAATTAAAACATATAGTATGGGCAAGCAGGGAGCTAGAACGATTCGCA
GTTAATCCTGGCCTGTTAGAAACATCAGAAGGCTGTAGACAAATACTGGG
ACAGCTACAACCATCCCTTCAGACAGGATCAGAAGAACTTAGATCATTAT
ATAATACAGTAGCAACCCTCTATTGTGTGCATCAAAGGATAGAGATAAAA
GACACCAAGGAAGCTTTAGACAAGATAGAGGAAGAGCAAAACAAAAGTAA
GACCACCGCACAGCAAGCGGCCGGCCGCTGATCTTCAGACCTGGAGGAGG
AGATATGAGGGACAATTAATTGGAGAAGTGAATTATATAAATATAAAGTA
GTAAAAATTGAACCATTAGGAGTAGCACCCACCAAGGCAAAGAGAAGAGT
GGTGCAGAGAGAAAAAAGAGCAGTGGGAATAGGAGCTTTGTTCCTTGGGT
TCTTGGGAGCAGCAGGAAGCACTATGGGCGCAGCGTCAATGACGCTGACG
GTACAGGCCAGACAATTATTGTCTGGTATAGTGCAGCAGCAGAACAATTT
GCTGAGGGCTATTGAGGCGCAACAGCATCTGTTGCAACTCACAGTCTGGG
GCATCAAGCAGCTCCAGGCAAGAATCCTGGCTGTGGAAAGATACCTAAAG
GATCAACAGCTCCTGGGGATTTGGGGTTGCTCTGGAAAACTCATTTGCAC
CACTGCTGTGCCTTGGAATGCTAGTTGGAGTAATAAATCTCTGGAACAGA
TTTGGAATCACACGACCTGGATGGAGTGGGACAGAGAAATTAACAATTAC
ACAAGCTTAATACACTCCTTAATTGAAGAATCGCAAAACCAGCAAGAAAA
GAATGAACAAGAATTATTGGAATTAGATAAATGGGCAAGTTTGTGGAATT
GGTTTAACATAACAAATTGGCTGTGGTATATAAAATTATTCATAATGATA
GTAGGAGGCTTGGTAGGTTTAAGAATAGTTTTTGCTGTACTTTCTATAGT
GAATAGAGTTAGGCAGGGATATTCACCATTATCGTTTCAGACCCACCTCC
CAACCCCGAGGGGACCCGACAGGCCCGAAGGAATAGAAGAAGAAGGTGGA
GAGAGAGACAGAGACAGATCCATTCGATTAGTGAACGGATCTCGACGGTA
TCGCCTTTAAAAGAAAAGGGGGGATTGGGGGGTACAGTGCAGGGGAAAGA
ATAGTAGACATAATAGCAACAGACATACAAACTAAAGAATTACAAAAACA
AATTACAAAAATTCAAAATTTTCGGGTTTATTACAGGGACAGCAGAGATC
CAGTTTATCTAATACGACTCACTATAGGGAGAGAGAGAGAATTACCCTCA
CTAAAGGGAGGAGAAGCATGAATTCTAGTAATCAATTACGGGGTCATTAG
TTCATAGCCCATATATGGAGTTCCGCGTTACATAACTTACGGTAAATGGC
CCGCCTGGCTGACCGCCCAACGACCCCCGCCCATTGACGTCAATAATGAC
GTATGTTCCCATAGTAACGCCAATAGGGACTTTCCATTGACGTCAATGGG
TGGAGTATTTACGGTAAACTGCCCACTTGGCAGTACATCAAGTGTATCAT
ATGCCAAGTCCGCCCCCTATTGACGTCAATGACGGTAAATGGCCCGCCTG
GCATTATGCCCAGTACATGACCTTACGGGACTTTCCTACTTGGCAGTACA
TCTACGTATTAGTCATCGCTATTACCATGCTGATGCGGTTTTGGCAGTAC
ACCAATGGGCGTGGATAGCGGTTTGACTCACGGGGATTTCCAAGTCTCCA
CCCCATTGACGTCAATGGGAGTTTGTTTTGGCACCAAAATCAACGGGACT
TTCCAAAATGTCGTAATAACCCCGCCCCGTTGACGCAAATGGGCGGTAGG
CGTGTACGGTGGGAGGTCTATATAAGCAGACGTCGTTTAGTGAACCGTCA
GATCACTAGATGCTTTATTGGATCCACAAGTTTGTACAAAAAAGCAGGCT
TGAAGGAATTCGGTACCATGGCGCCCCGAAGCCTCCTCCTGCTGCTCTCA
GGGGCCCTGGCCCTGACCGATACTTGGGCAGGCTCCCACTCCTTGAGGTA
TTTCAGCACCGCTGTGTCGCGGCCCGGCCGCGGGGAGCCCCGCTACATCG
CCGTGGAGTACGTAGACGACACGCAATTCCTGCGGTTCGACAGCGACGCC
GCGATTCCGAGGATGGAGCCGCGGGAGCCGTGGGTGGAGCAAGAGGGGCC
GCAGTATTGGGAGTGGACCACAGGGTACGCCAAGGCCAACGCACAGACTG
ACCGAGTGGCCCTGAGGAACCTGCTCCGCCGCTACAACCAGAGCGAGGCT
GGGTCTCACACCCTCCAGGGAATGAATGGCTGCGACATGGGGCCCGACGG
ACGCCTCCTCCGCGGGTATCACCAGCACGCGTACGACGGCAAGGATTACA
TCTCCCTGAACGAGGACCTGCGCTCCTGGACCGCGGCGGACACCGTGGCT
CAGATCACCCAGCGCTTCTATGAGGCAGAGGAATATGCAGAGGAGTTCAG
GACCTACCTGGAGGGCGAGTGCCTGGAGTTGCTCCGCAGATACTTGGAGA
ATGGGAAGGAGACGCTACAGCGCGCAGATCCTCCAAAGGCACACGTTGCC
CACCACCCCATCTCTGACCATGAGGCCACCCTGAGGTGCTGGGCCCTGGG
CTTCTACCCTGCGGAGATCACGCTGACCTGGCAGCGGGATGGGGAGGAAC
AGACCCAGGACACAGAGCTTGTGGAGACCAGGCCTGCAGGGGATGGAACC
TTCCAGAAGTGGGCCGCTGTGGTGGTGCCTTCTGGAGAGGAACAGAGATA
CACATGCCATGTGCAGCACGAGGGGCTGCCCCAGCCCCTCATCCTGAGAT
GGGAGCAGTCTCCCCAGCCCACCATCCCCATCGTGGGCATCGTTGCTGGC
CTTGTTGTCCTTGGAGCTGTGGTCACTGGAGCTGTGGTCGCTGCTGTGAT
GTGGAGGAAGAAGAGCTCAGATAGAAACAGAGGGAGCTACTCTCAGGCTG
CAGCCTACTCAGTGGTCAGCGGACTCTTGATGATAACATGGTGGTCAAGC
TTATTTCTCCTGGGGGTGCTCTTCCAAGGATATTTGGGCTGCCTCCGGAG
TCACAGTGTCTTGGGCCGCCGGAAGGTGGGTGACATGTGGATCTTGTTTT
TTTTGTGGCTGTGGACATCTTTCAACACTGCCTTCTTGGCCTTGCAAAGC
CTTCGCTTTGGCTTCGGCTTTAGGAGGGGCAGGAGCTTCCTTCTTCGTTC
TTGGCACCATCTTATGAAAAGGGTCCAGATTAAGATTTTTGACTAGCTCG
AGTGCGGCCGC

In one embodiment, the invention encompasses isolated or substantially purified nucleic acids. An “isolated” nucleic acid is free of other nucleic acids with which it is typically associated in nature, other than those identified by its sequence identification number. A “purified” nucleic acid molecule is substantially free of chemical precursors or other chemicals when chemically synthesized, or is substantially free of cellular material if made by recombinant DNA techniques. In various embodiments described herein, the nucleic acids for use in the methods, compounds, compositions, and uses described herein may be double-stranded (e.g., antisense RNAs) or single-stranded, but the nucleic acids are typically single-stranded.

In another embodiment, the nucleic acid described herein is provided in a sterile container (e.g., a vial) or package, for example, an ampoule or a sealed vial. In another illustrative aspect, a nucleic acid described herein can have “a” sequence consisting of, or can have “the” sequence consisting of, a nucleic acid sequence selected from the group consisting of SEQ ID NO: 1 and SEQ ID NO: 2. In other aspects, the nucleic acid described herein can “comprise” or “consist of” a nucleic acid sequence selected from the group consisting of SEQ ID NO: 1 and SEQ ID NO: 2. In another embodiment, the nucleic acid described herein can be synthetic.

In one illustrative embodiment, the nucleic acids for use in the methods, uses, compounds, and compositions described herein can be modified by substitution, deletion, truncation, and/or can be fused with other nucleic acid molecules wherein the resulting nucleic acids hybridize specifically under highly stringent conditions to the complements of nucleic acids of SEQ ID NO: 1 or SEQ ID NO: 2, and wherein the modified nucleic acids are useful in the methods or uses described herein. Derivatives can also be made such as phosphorothioate, phosphotriester, phosphoramidate, and methylphosphonate derivatives (Goodchild, et al., Proc. Natl. Acad. Sci. 83:4143-4146 (1986), incorporated herein by reference).

In another embodiment, nucleic acid molecules are provided having about 60%, about 70%, about 75%, about 80%, about 85%, about 90%, about 95%, about 96%, about 97%, about 98%, or about 99% homology to SEQ ID NO: 1 or SEQ ID NO: 2. Determination of percent identity or similarity between sequences can be done, for example, by using the GAP program (Genetics Computer Group, software; now available via Accehrys on http://www.accelrys.com), and alignments can be done using, for example, the ClustalW algorithm (VNTI software, InforMax Inc.). A sequence database can be searched using the nucleic acid sequence of interest. Algorithms for database searching are typically based on the BLAST software (Altschul et al., 1990). In some embodiments, the percent identity can be determined along the full-length of the nucleic acid.

Techniques for synthesizing the nucleic acids described herein, such as nucleic acids of SEQ ID NO: 1 or SEQ ID NO: 2, or fragments thereof, are well-known in the art and include chemical syntheses. Such techniques are described in Sambrook et al., “Molecular Cloning: A Laboratory Manual”, 3rd Edition, Cold Spring Harbor Laboratory Press, (2001), incorporated herein by reference. In one embodiment, nucleic acids for use in the methods described herein can be made commercially and can be obtained from, for example, Ambion Inc. (Austin, Texas), Darmacon Inc. (Lafayette, Colorado), or InvivoGen (San Diego, California). Techniques for purifying or isolating the nucleic acids described herein are well-known in the art. Such techniques are described in Sambrook et al., “Molecular Cloning: A Laboratory Manual”, 3rd Edition, Cold Spring Harbor Laboratory Press, (2001), incorporated herein by reference.

In one aspect, the compounds described herein can be in the form of a pharmaceutical composition. In another embodiment, uses of these pharmaceutical compositions for the manufacture of a medicament for treating amyotrophic lateral sclerosis are provided. In yet other embodiments, the pharmaceutical compositions are provided for use in treating amyotrophic lateral sclerosis.

In one embodiment, the compounds described herein for inducing expression of the MHC class I molecule, HLA-F, in motor neurons may be administered as a formulation in association with one or more pharmaceutically acceptable carriers. The carriers can be excipients. The choice of carrier will to a large extent depend on factors such as the particular mode of administration, the effect of the carrier on solubility and stability, and the nature of the dosage form. Pharmaceutical compositions suitable for the delivery of the compound, or additional therapeutic agents to be administered with the compound, and methods for their preparation will be readily apparent to those skilled in the art. Such compositions and methods for their preparation may be found, for example, in Remington: The Science & Practice of Pharmacy, 21st Edition (Lippincott Williams & Wilkins, 2005), incorporated herein by reference.

In one embodiment, a pharmaceutically acceptable carrier may be selected from any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions, and sterile powders for the preparation of sterile injectable solutions or dispersions. Supplementary active compounds can also be incorporated into the pharmaceutical compositions of the invention.

In various embodiments, liquid formulations may include suspensions and solutions. Such formulations may comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, such as a lyophilizate. Thus, in one embodiment, the lyophilizate can be a reconstitutable or a reconstituted lyophilizate.

In one illustrative aspect, an aqueous suspension may contain the active materials (i.e., a nucleic acid comprising or consisting of SEQ ID NO: 1 or SEQ ID NO: 2) in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents. In other embodiments, isotonic agents, for example, sugars, polyalcohols such as mannitol, sorbitol, or sodium chloride can be included in the pharmaceutical composition.

In one embodiment the excipient comprises a buffer. In one embodiment, the pH of the buffer is about 5.0 to about 8.0. The buffer may be any acceptable buffer for the indicated pH range and physiological compatibility. In addition a buffer may additionally act as a stabilizer. In one embodiment, the buffer comprises an ascorbate, sorbate, formate, lactate, fumarate, tartrate, glutamate, acetate, citrate, gluconate, histidine, malate, phosphate or succinate buffer.

In one aspect, a compound (i.e., a drug, a peptide, or a nucleic acid), or additional therapeutic agent as described herein, may be administered directly into the blood stream, into muscle, or into an internal organ. Suitable routes for parenteral administration include intravenous, intraarterial, intraperitoneal, intrathecal, epidural, intracerebroventricular, intrasternal, intracranial, intramuscular, intraosseous, intraocular, and subcutaneous delivery. In other embodiments, lumbar puncture or cisterna magna administration can be used. In yet another embodiment, the compound can be delivered to the brain, the spinal cord, the central nervous system, or the peripheral nervous system of the patient. In other aspects, the compound can be delivered to an upper or lower motor neuron of the patient.

In one embodiment, suitable means for parenteral administration include needle (including microneedle) injectors, needle-free injectors and infusion techniques. Examples of parenteral dosage forms include aqueous solutions of the active agent, in an isotonic saline, glucose (e.g., 5% glucose solutions), or other well-known pharmaceutically acceptable liquid carriers such as liquid alcohols, glycols, esters, and amides. Prolonged absorption of the injectable compositions can be brought about by including in the composition an agent which delays absorption, for example, a monostearate salt.

In another embodiment, the compound described herein may be in the form of a kit. In one aspect, the compound can be a nucleic acid and the nucleic acid can comprise a vector. In another illustrative aspect, the nucleic acid can comprise SEQ ID NO: 1 or SEQ ID NO: 2. In one embodiment, the compound is in a sterile container (e.g., a vial) or package, for example, an ampoule or a sealed vial in the kit. In this embodiment, the compound in the kit can be in the form of a reconstitutable lyophilizate. In another embodiment, the kit can contain instructions for use of the compound for treating a patient with amyotrophic lateral sclerosis.

In another embodiment, any of the preceding kit embodiments wherein the dose of the compound in the pharmaceutical composition is in the range of 1 to 5 μg/kg is described. In another embodiment, any of the preceding kit embodiments wherein the dose of the compound in the pharmaceutical composition is in the range of 1 to 3 μg/kg is described.

In another embodiment, the kit of any of the preceding kit embodiments is described wherein the purity of the compound is at least 90% based on weight percent. In another embodiment, the kit of any of the preceding embodiments is described wherein the purity of the compound is at least 95% based on weight percent. In another embodiment, the kit of any of the preceding embodiments is described wherein the purity of the compound is at least 96% based on weight percent. In another embodiment, the kit of any of the preceding embodiments is described wherein the purity of the compound is at least 97% based on weight percent. In another embodiment, the kit of any of the preceding kit embodiments is described wherein the purity of the compound is at least 98% based on weight percent. In another embodiment, the kit of any of the preceding kit embodiments is described wherein the purity of the compound is at least 99% based on weight percent. In another embodiment, the kit of any of the preceding embodiments is described wherein the purity of the compound is at least 99.5% based on weight percent.

In another illustrative aspect, the kit of any of the preceding kit embodiments is described wherein the compound or the composition is in a parenteral dosage form. The parenteral dosage form can be selected from the group consisting of an intradermal dosage form, a subcutaneous dosage form, an intramuscular dosage form, an intraperitoneal dosage form, an intravenous dosage form, an intracranial dosage form, an intraosseous dosage form, an intraocular dosage form, an introcerebroventricular dosage form, and an intrathecal dosage form.

In yet another embodiment, the kit can comprise the composition and the composition can further comprise a pharmaceutically acceptable carrier. In one embodiment, the pharmaceutically acceptable carrier can be a liquid carrier selected from the group consisting of saline, glucose, alcohols, glycols, esters, amides, and a combination thereof.

Any effective regimen for administering the composition or the compound can be used. For example, the composition or the compound can be administered as a single dose, or can be divided and administered as a multiple-dose daily regimen. Further, a staggered regimen, for example, one to five days per week can be used as an alternative to daily treatment, and for the purpose of the pharmaceutical compositions, kits, methods, and uses described herein, such intermittent or staggered daily regimen is considered to be equivalent to every day treatment and is contemplated. In one illustrative embodiment the patient is treated with multiple injections of the composition or the compound to eliminate the disease state (i.e., amyotrophic lateral sclerosis) or to reduce or stabilize the symptoms of disease. In one embodiment, the patient is injected multiple times (preferably about 2 up to about 50 times), for example, at 12-72 hour intervals or at 48-72 hour intervals. Additional injections of the compound can be administered to the patient at an interval of days or months after the initial injections(s), and the additional injections can prevent recurrence of the disease or can prevent an increase in the severity of the symptoms of disease.

In one embodiment, administration of the compounds and compositions described herein according to the methods and uses of the invention may increase the survival of the patient by 90 days or greater. In another embodiment, administration of the compounds and compositions described herein according to the methods and uses of the invention may increase the survival of the patient by at least 20 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 90 days, at least 95 days, at least 100 days, at least 150 days, at least 200 days, at least 250 days, or at least 300 days as compared to a patient who does not receive the treatment described herein.

In one aspect, the unitary daily dosage of the compound can vary significantly depending on the patient condition, the disease state being treated, the purity of the compound and its route of administration and tissue distribution, and the possibility of co-usage of other therapeutic treatments. The effective amount to be administered to a patient is based on body surface area, mass, and physician assessment of patient condition. Effective doses can range, for example, from about 1 ng/kg to about 1 mg/kg, from about 1 μg/kg to about 500 μg/kg, and from about 1 μg/kg to about 100 μg/kg. These doses are based on an average patient weight of about 70 kg, and the kg are kg of patient body weight (mass). In one embodiment, the compound or pharmaceutical composition is in a multidose form. In another embodiment, the compound or pharmaceutical composition is a single dose form (i.e., a unit dose form or a dosage unit). “Effective doses” are doses that eliminate, alleviate, or reduce at least one symptom of amyotrophic lateral sclerosis or slow progression or prevent progression of amyotrophic lateral sclerosis or prolong survival of a patient with amyotrophic lateral sclerosis.

In one embodiment, the compound can be administered in a dose of from about 1.0 ng/kg to about 1000 μg/kg, from about 10 ng/kg to about 1000 μg/kg, from about 50 ng/kg to about 1000 μg/kg, from about 100 ng/kg to about 1000 μg/kg, from about 500 ng/kg to about 1000 μg/kg, from about 1 ng/kg to about 500 μg/kg, from about 1 ng/kg to about 100 μg/kg, from about 1 μg/kg to about 50 μg/kg, from about 1 μg/kg to about 10 μg/kg, from about 5 μg/kg to about 500 μg/kg, from about 10 μg/kg to about 100 μg/kg, from about 20 μg/kg to about 200 μg/kg, from about 10 μg/kg to about 500 μg/kg, or from about 50 μg/kg to about 500 μg/kg. The total dose may be administered in single or divided doses and may, at the physician's discretion, fall outside of the typical range given herein. These dosages are based on an average patient weight of about 70 kg and the “kg” are kilograms of patient body weight. The physician will readily be able to determine doses for subjects whose weight falls outside this range, such as infants and the elderly.

In another embodiment, the compound can be administered at a dose of from about 1 μg/m² to about 500 mg/m², from about 1 μg/m² to about 300 mg/m², or from about 100 μg/m² to about 200 mg/m². In other embodiments, the compound can be administered at a dose of from about 1 mg/m² to about 500 mg/m², from about 1 mg/m² to about 300 mg/m², from about 1 mg/m² to about 200 mg/m², from about 1 mg/m² to about 100 mg/m², from about 1 mg/m² to about 50 mg/m², or from about 1 mg/m² to about 600 mg/m². The total dose may be administered in single or divided doses and may, at the physician's discretion, fall outside of the typical range given herein. These dosages are based on m² of body surface area.

In another embodiment where a viral vector is used, the titer may vary depending on the mode of administration, the patient weight, etc. and may be about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵ or about 1×10¹⁶ DNase resistant particles per ml. In another embodiment where a viral vector is used, the dosages administered may be about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵ or about 1×10¹⁶ viral genomes per kilogram of patient body weight. These dosages are based on an average patient weight of about 70 kg and the “kg” are kilograms of patient body weight.

In another embodiment, the pharmaceutical compositions and/or dosage forms of the compound for administration are prepared from compounds with a purity of at least about 90%, or at least about 95%, or at least about 96%, or at least about 97%, or at least about 98%, or at least about 99%, or at least about 99.5%. In another embodiment, pharmaceutical compositions and/or dosage forms of the compound for administration are prepared from compounds with a purity of at least 90%, or at least 95%, or at least 96%, or at least 97%, or at least 98%, or at least 99%, or at least 99.5%. The purity of the compound may be measured using any conventional technique, including various chromatography or spectroscopic techniques, such as high pressure or high performance liquid chromatography, nuclear magnetic resonance spectroscopy, TLC, UV absorbance spectroscopy, fluorescence spectroscopy, and the like.

As used herein, purity determinations may be based on weight percentage, mole percentage, and the like. In addition, purity determinations may be based on the absence or substantial absence of certain predetermined components. It is also to be understood that purity determinations are applicable to solutions of the compounds and pharmaceutical compositions prepared by the methods described herein. In those instances, purity measurements, including weight percentage and mole percentage measurements, are related to the components of the solution exclusive of the solvent.

In another embodiment, the compound or the pharmaceutical composition is provided in a sterile container (e.g., a vial) or package, for example, an ampoule or a sealed vial.

In another embodiment, the methods, pharmaceutical compositions, compounds, uses, and kits, described herein include the following examples. The examples further illustrate additional features of the various embodiments of the invention described herein. However, it is to be understood that the examples are illustrative and are not to be construed as limiting other embodiments of the invention described herein. In addition, it is appreciated that other variations of the examples are included in the various embodiments of the invention described herein.

### Example 1

### Animals

All procedures were performed in accordance with the NIH Guidelines on the care and use of vertebrate animals and approved by the Institutional Animal Care and Use Committee of the Research Institute at Nationwide Children's Hospital. Transgenic mice that expressed human SOD1 carrying the G93A mutation (B6SJL-TgSOD1^{G93A}), referred to here as SOD1^{G93A} mice, were obtained from Jackson Laboratories and maintained, characterized by the guidelines of Jackson Laboratory for the entire of animal study (Bar Harbor, ME). Animals were housed under light/dark (12:12 hour) cycle with food and water ad libitum. At each generation, animals were genotyped, SOD1^{G93A} transgene copy number were verified by quantitative PCR, prior to either the isolation of primary cells or the injection of AAV9. To minimize variability due to gender effects on survival and behavior analysis, only female mice were used for AAV9-H2K injection experiments. After confirming genotype, SOD1^{G93A} animals were randomly selected for AAV9 injections of control, H2D or H2K. In each litter, half of the animals were treated with AAV9-empty and half with AAV9-H2K. All procedures were performed in accordance with the NIH Guidelines and were approved by the Nationwide Children's Research Institutional Animal Care and Use Committee.

### Example 2

### SOD1^{G93A} Mouse Survival and Behavior Analysis

Disease stages (previously described in Frakes, A. E., et al. Microglia induce motor neuron death via the classical NFkappaB pathway in amyotrophic lateral sclerosis. Neuron, 81, 1009-1023 (2014); Foust, K. D., et al. Therapeutic AAV9-mediated suppression of mutant SOD1 slows disease progression and extends survival in models of inherited ALS. Mol Ther, 21, 2148-2159 (2013)) included the following: “Pre-symptomatic stage,” during which mice displayed no disease symptoms and were not yet at peak body weight; “Symptomatic-stage,” during which mice showed overt symptoms characterized by tremors and hindlimb paralysis and showed a 10% or more decrease from the peak of body weight; “End-stage,” during which animals exhibited forelimb and hindlimb paralysis and were unable to right themselves within 30 seconds after being placed on its back. “Disease onset” was defined as the age at which mice reach their peak body weight. “Disease progression” was defined as the time period between disease onset and end stage. Motor coordination was recorded using a rotarod instrument (Columbus Instruments, Columbus, OH). Three trials were performed on accelerating rotarod beginning at 5 rpm/minutes twice a week. The time each mouse remained on the rod was recorded. Analysis of the data was performed blindly but not randomly.

### Example 3

### Isolation and Culture of Mouse Glial Cells

Astrocytes and microglia were isolated from 110-130 day old SOD1^{G93A} and wild-type B6SJL mice. Astrocyte cultures were prepared as previously described with minor modifications (Noble, M. & Mayer-Proschel, M. Culture of astrocytes, oligodendrocytes, and O-2A progenitor cells, (MIT press, Cambridge, 1998). Briefly, spinal cords were enzymatically dissociated to single cells with a mixture of Papain (2.5 U/ml; Worthington Biochemical, Lakewood, NJ), Dispase grade II (1 U/ml; Boehringer Mannheim Corporation, Indianapolis, IN) and Dnase I (250 U/ml; Worthington Biochemical) for about 20 minutes. After filtration with a 70 μm nylon mesh, cells were pelleted, and resuspended in DMEM/F12 (Invitrogen, Carlsbad, CA) which was supplemented with 10% fetal bovine serum (FBS, Invitrogen) and 0.2% N2 supplement (Invitrogen). The cells were then plated onto laminin coated 75 cm2 tissue culture flasks. Upon confluence, flasks were shaken overnight in order to remove potential microglial cells and then were treated with cytosine arabinose (20 PM, Sigma-Aldrich, St. Louis, MO). Prior to use, astrocyte preparations were screened for the presence of cytotoxic T-lymphocytes (CTLs) and natural killer (NK) cells and were found to be devoid of them.

Microglia were isolated following a protocol previously described (Frakes, A. E., et al. Microglia induce motor neuron death via the classical NFkappaB pathway in amyotrophic lateral sclerosis. Neuron, 81, 1009-1023 (2014). Briefly, tissues were fragmented with a scalpel and incubated in enzymatic solution containing papain (2.5 U/ml; Worthington Biochemical) for 60 minutes at 37° C. 20% FBS in Hank's Balanced Salt Solution (HBSS, Invitrogen) was applied to the tissue, and they were then centrifuged at 200×g for 4 minutes. Cell pellets were resuspended in 2 ml of DNase I (0.5 mg/ml, Worthington Biochemical) in HBSS and were incubated for 5 minutes at room temperature. Tissue was gently disrupted with fire-polished Pasteur pipettes, filtered through a 70 micron cell strainer, and centrifuged at 200×g for 4 minutes. Pellet was then resuspended in 20 ml of 20% isotonic Percoll (GE healthcare) in HBSS. 20 ml of pure HBSS was carefully laid on top the percoll layer and centrifugation was performed at 200×g for 20 minutes with slow acceleration and no brake. The pellet containing the mixed glial cell population was washed once with HBSS and was suspended in Dulbecco's modified Eagle's/F12 medium with GlutaMAX™ (DMEM/F12, Invitrogen) supplemented with 10% heat inactivated FBS, antibiotic-antimycotic (all from Life Technologies) and 5 ng/ml of carrier-free murine recombinant granulocyte and macrophage colony stimulating factor (GM-CSF) (R&D systems, Minneapolis, MN). Cell suspension was then plated on a poly-L-lysine (Sigma) coated plate and maintained at 37° C. The media was replaced every 3 days until the cells reached confluency. Microglia that formed a non-adherent, floating cell layer were collected, replated, and cultured for an extended period of time. Microglia were incubated for 3 days without GM-CSF before being re-plated for co-culture with MNs. Prior to analysis, microglia preparations were tested for the presence of CTLs and NK cells and were found to be devoid of them.

### Example 4

### Mouse NPC Isolation and Differentiation into Astrocytes

NPCs were isolated according to methods previously described (Miranda, C. J., et al. Aging brain microenvironment decreases hippocampal neurogenesis through Wnt-mediated survivin signaling. Aging Cell (2012); Ray, J. & Gage, F. H. Differential properties of adult rat and mouse brain-derived neural stem/progenitor cells. Mol Cell Neurosci, 31, 560-573 (2006). Briefly, spinal cords were enzymatically dissociated in the same way as described for astrocytes. The cell suspension obtained was mixed with an equal volume of isotonic Percoll (GE Healthcare) and was centrifuged at 20,000×g for 30 minutes at room temperature. Cells from the low-buoyancy fraction (5-10 ml above the red blood cell layer) were harvested, washed thoroughly with D-PBS/PSF (Invitrogen) and plated in 60 mm uncoated plates. Cells were grown in growth medium (DMEM/F12, Invitrogen) with 1% N2 supplement (Invitrogen), 20 ng/ml of fibroblast growth factor-2 (FGF-2, Peprotech, Rocky Hill, NJ) and 20 ng/ml of endothelial growth factor (EGF, Peprotech). Cells were first grown as neurospheres and then were placed on a polyornithine-laminin (P/L)-coated plates, in which they grow as monolayer cultures. NPC cultures were found to be devoid of astrocytes, microglia, CTLs and NK cells contaminants. Once cultures were established, NPCs from wild-type and SOD1^{G93A} mice were used to generate astrocytes by withdrawing growth factors and supplementing the medium with 10% FBS (astrocyte media). The media was changed every 2 days thereafter. Astrocytes were allowed to mature for 7 days prior to being used in the experiments described above. Highly enriched astrocyte cultures were obtained with no detectable levels of microglia, CTLs and NK cells.

### Example 5

### Human Post-Mortem NPC Derived Astrocytes

Post-mortem spinal cords were obtained from the National Disease Research Interchange (NDRI, Philadelphia, PA) and from Dr. Fred Gage (Salk Institute, CA). Informed consents were obtained from all subjects. Receipt of human tissues was granted through Nationwide Children's Hospital Institutional Review Board (IRB08-00402) and all human samples were used in accordance with their approved protocols. Extensive phenotypic characterization of the cell lines used herein has been previously described (Haidet-Phillips, A. M., et al. Astrocytes from familial and sporadic ALS patients are toxic to motor neurons. Nat Biotechnol, 29, 824-828 (2011); Meyer, K., et al. Direct conversion of patient fibroblasts demonstrates non-cell autonomous toxicity of astrocytes to motor neurons in familial and sporadic ALS. Proc Nat Acad Sci USA, 111, 829-832 (2014)). A summary of the demographic information associated with NPC derived astrocyte cell lines is shown in Table 4. Cells were grown on laminin-coated plates in astrocyte media supplemented 0.2% N2 supplement (Invitrogen). Media change occurred every 3 days, and cells were passaged when cultures reached 80% confluency. Human astrocyte cultures were found to be devoid of microglia, CTLs and NK cells.

TABLE 4
Summary of the demographic information
associated with NPC derived astrocyte lines.

| | | Age | | Time from |
|---|---|---|---|---|
| ID | Diagnosis | (yr.) | Gender | Diagnosis to Death |
| Non-ALS #1 | Non-ALS | 61 | Male | N/A |
| Non-ALS #2 | Non-ALS | N/A | N/A | N/A |
| Non-ALS #3 | Non-ALS | 87 | Male | N/A |
| fALS #1 ^{*} | Familial ALS | 57 | Male | 8 Months |
| sALS #1 | Sporadic ALS | 70 | Male | 20 Months |
| sALS #2 | Sporadic ALS | 55 | Male | 14 Months |
| sALS #3 | Sporadic ALS | 64 | Male | 14 Months |
| sALS #4 | Sporadic ALS | 70 | Female | 60 Months |
| sALS #5 | Sporadic ALS | 67 | Male | 9 Months |

N/A, non-available;
* sequencing results confirmed A4V mutation in the SOD1 locus.

### Example 6

### iPSC Generation

NPCs, expressing the MN Hb9::GFP reporter, obtained from wild-type and SOD1^{G93A} mice were converted to iPSCs. As previously described, retrovirus encoding OCT3/4 and KLF4 were sufficient to generate iPSC clones (Hester, M. E., et al. Two factor reprogramming of human neural stem cells into pluripotency. PLoS One, 4, e7044 (2009); Kim, J. B., et al. Pluripotent stem cells induced from adult neural stem cells by reprogramming with two factors. Nature, 454, 646-650 (2008)). 20 viral particles per cell were needed to efficiently reprogram the cells. Cells were cultured in the presence of NPC media for four days followed by a change to mouse embryonic stem cell (mESC) media with DMEM (Millipore, Billerica, MA), supplemented with 18% ES FBS (Invitrogen), L-glutamine (2 mM, Invitrogen), nonessential amino acids (1×, Millipore), antibiotic-antimycotic (Invitrogen), 2-mercaptoethanol (Sigma), and recombinant LIF (100 U/ml, Millipore). iPSC clones were morphologically similar to mouse ESCs (HBG3 cells, Thomas Jessell, Columbia University) and were obtained within two weeks. A wide panel of markers was used to compare ESCs with the newly generated iPSC lines.

### Example 7

### Mouse MN Differentiation

Mouse ESCs or iPSCs expressing Hb9::GFP reporter were cultured on top of inactivated mouse fibroblasts (Millipore). MN differentiation was induced by plating 1-2×106 mES cells per 10 cm dish in the presence of 2 μM retinoic acid (Sigma-Aldrich) and 2 μM purmorphamine (Calbiochem, Billerica, MA). After 5 days of differentiation, embryonic bodies were dissociated and sorted based on levels of GFP using a FACSVantage/DiVa sorter (BD Biosciences, Rockville, MD).

### Example 8

### NPC Differentiation into GABAergic Neurons

Mouse NPCs were induced to differentiate into GABAergic neurons by supplementing growth medium with 0.1% FBS (Invitrogen), retinoic acid (1 μM, Sigma-Aldrich), and forskolin (5 μM, Sigma-Aldrich). Media were changed every day. Cultures were allowed to differentiate for 7 days prior to being used for experiments.

### Example 9

### Co-Culture of Mouse Astrocytes with Mouse MNs

Astrocytes were plated at the density of 35,000 cells per well in 96-well plates coated with laminin. After 48 hours, FACS sorted GFP+ MNs were plated on top of the astrocyte monolayer at a density of 10,000 cells per well. Co-cultures were performed in MN media composed of DMEM/F12 (Invitrogen) supplemented with 5% horse serum (Equitech Bio, Kerrville, TX), 2% N2 supplement (Invitrogen), 2% B27 supplement (Invitrogen), 10 ng/ml GDNF (Invitrogen), 10 ng/ml BDNF (Invitrogen), 10 ng/ml CNTF (Invitrogen). Half of the media was replaced every other day, with the addition of fresh growth factors.

### Example 10

### Sustained Expression of MHCI Molecules in Mouse MNs

To express histocompatibility 2 subclasses in MNs, a previously described protocol was followed, with minor modifications (Kaech, S. & Banker, G. Culturing hippocampal neurons. Nat Protoc, 1, 2406-2415 (2006)). Briefly, wild-type astrocytes were plated on a laminin-coated transwell (Corning, Lowell, MA) using MN media. After 24 hours, sorted GFP+ MNs were plated on a separate laminin-coated 96 well plate in media, conditioned by wild-type astrocytes. Four hours later, the transwell containing wild-type astrocytes was transferred into the MN plate, after verification that all MNs were fully attached and were starting to show neuritic extensions. The following day, the transwell of wild-type astrocytes was removed and the MNs were infected with Lv-H2K, H2D or H2L (40 viral particles per MN). Twelve hours post-infection, co-culture with wild-type astrocytes via transwell was resumed. After 72 hours, the transwell was removed and the co-culture experiments with wild-type and SOD1^{G93A} astrocytes were initiated. Experiments were performed independently by two investigators.

### Example 11

### Astrocyte Conditioned Media

Astrocyte conditioned medium was prepared by co-culturing mouse MNs and mouse astrocytes for 120 hours. After removal of cell debris by centrifugation (500×g for 10 min), medium was supplemented with GDNF, CNTF and BDNF. This medium was added to MNs cultures and cultures were evaluated after 24 hours.

### Example 12

### Co-Culture of Human Astrocytes with Human MNs Expressing HLA-F

MNs were obtained by differentiating human ES cell-derived MN progenitors (Lonza, Walkersville, MD) following the manufacturer's instructions. MN progenitors were plated at a density of 10,000 cells per well in a laminin coated 96-well plate. 48 hours after plating, the cells were infected with adenovirus encoding Ngn2, Isl1, and Lhx3 in order to enhance efficiency and shorten the time required for MN differentiation. After 10 days of MN differentiation, MNs were infected with lentivirus to overexpress HLA-F (20 viral particles per MN). 3 days after, 10,000 human astrocytes were added to each well. Co-cultures were allowed to continue for another 14 days, with half of the media being replaced every other day. Due to the limited number of MNs available at a time of study, astrocytes were randomly chosen and co-culture initiated.

### Example 13

### Viral Vectors

To knockdown H2-K^{b} levels in MNs or GABAergic neurons, sequences from the RNAi Consortium lentiviral shRNA library were screened and the sequence 5′-TAAAGAGAACTGAGGGCTCTG-3′ (SEQ ID NO: 3) was used. The sequence 5′-GGCGTAGATGTCCGATAAGAA-3′ (SEQ ID NO: 4) was used for the scrambled shRNA control. The cDNAs of histocompatibility 2 subclasses were obtained and cloned into a lentiviral vector. H2-K^{b} cDNA in a viral vector was purchased from Genecopia (Rockville, MD) referred to as H2K; H2-D^{b} cDNA (NM_010380.3) was purchased from Thermoscientific (Pittsburgh, PA) referred to as H2D; H2-L^{d} cDNA (NM_001267808.1) was synthesized by Genscript (Piscataway, NJ) referred to as H2L. To knockdown Kir3dl2 gene in human ALS astrocytes, sequences from the RNAi Consortium lentiviral shRNA library were also screened and the sequence 5′-TAAAGGAGAAAGAAGAGGAGG-3′ (SEQ ID NO: 5) was used. The sequence 5′-GGGAGAAAGAAGGAGGATAAA-3′ (SEQ ID NO: 6) was used for the scrambled shRNA control. The HLA-F cDNA (NM_001098479.1) was purchased from Genecopia (Rockville). The production and purification of the lentivirus were performed as previously reported.

### Example 14

### MN Cell Viability

At various time points during the co-culture of mouse astrocytes and mouse MNs, cell survival, neuritic length and soma size of MNs were recorded using a fully automated IN CELL 6000 cell imager (GE Healthcare). Images were processed with the Developer and Analyzer software package (GE Healthcare). Otherwise noted, images shown represent 120 hours post co-culture. All counts were performed in triplicate and repeated at least three times.

### Example 15

### AAV9 Injection in SOD1^{G93A} Mice

H2-K^{b} or H2-D^{b} cDNA sequence used in our in vitro experiments was cloned into a AAV9 vector that has been reported to transduce high levels of MNs in brain and spinal cords (Foust, K. D., et al. Intravascular AAV9 preferentially targets neonatal neurons and adult astrocytes. Nat Biotechnol, 27, 59-65 (2009); Foust, K. D., et al. Therapeutic AAV9-mediated suppression of mutant SOD1 slows disease progression and extends survival in models of inherited ALS. Mol Ther, 21, 2148-2159 (2013)). Self-complementary AAV9 encoding no transgene (AAV9-empty), or GFP (AAV9-GFP) or H2-D^{b} (AAV9-H2D) or H2-K^{b} (AAV9-H2K) was produced by transient transfection procedures using a double-stranded AAV2-ITR-based CB vector, with a plasmid encoding Rep2Cap9 sequence as previously described along with an adenoviral helper plasmid pHelper (Stratagene, Santa Clara, CA) in 293 cells. Injections of AAV9 were performed directly to the cerebral spinal fluid (CSF) at postnatal day 1 by direct injection into the lateral ventricles. Animals received a total dose of 2.33×10¹³ vg/kg. To validate and minimize variability associated with the injection procedure, at least two fold (24) of the minimum number of animals that the guidelines for preclinical animal research in ALS/MND suggests was aimed for the survival studies.

### Example 16

### RNA Isolation and RT-PCR

RNA was harvested using the RT² q-PCR-grade RNA isolation kit (Qiagen, Frederick, MD) and total RNA was reverse transcribed with RT² First Strand Kit (Qiagen) according to the manufacturer's instructions. After ensuring all cDNAs were devoid of genomic DNA contamination, mouse and human gene transcripts were amplified using gene-specific primers described in Table 5. For detection of MHCI inhibitor receptor transcripts (Ly49 or human killer-cell immunoglobulin-like receptor transcripts (KIRs)), astrocytes were prepared by co-culturing with mouse MNs and RT-PCR was performed using primer sets previously described (Thompson, A., van der Slik, A. R., Koning, F. & van Bergen, J. An improved RT-PCR method for the detection of killer-cell immunoglobulin-like receptor (KIR) transcripts. Immunogenetics, 58, 865-872 (2006)). Real-time quantitative PCR reactions were performed using RT² Real-Time SYBR Green/Rox Master Mix (Qiagen, Frederick, MD). Each sample was run in triplicate and relative concentration was calculated using the ddCt values normalized to endogenous actin transcript.

TABLE 5
List of primers used for RT-PCR analysis.

| | | | | Product | |
|---|---|---|---|---|---|
| Species | Gene | Forward Primer (5′-3′) | Reverse Primer (5′-3′) | Size (bp) | References |
| | [symbol] | TCCCACGATGAGTGAGCCA | TACCTTTAACTCTAGTTGGAAAA | [symbol] | [symbol] |
| | [symbol] | GATGAATGAGCCGGAGGTC | TTTCACTGTTCATCTGTCCT | 369 | [symbol] |
| | [symbol] | GGTGAGGCTTGAGGAGACAG | CTTCCCACAAATACAGATGAATGAT | 710 | [symbol] |
| | | | TTATCACATTTATC | | [symbol] |
| | [symbol] | GGATTGACAATCACCCATCTAAG | GAGAACATTCCAAAAATCTTCAG | 318 | |
| | [symbol] | GAGACAGGGAAGCCTCAAAAAG | TGGTGCTGCACTTATCGTGG | 193 | |
| | [symbol] | TACTCAGCAGACCTTGAACCT | CAGTCTTGGCAGCAAGTTGAC | 307 | |
| | [symbol] | ATGAACGCTACACACTGCATC | CCATCCTTTTGCCAGTTCCTC | 182 | |
| | [symbol] | CAGCCCATGAATTACCCATGT | ATTTGTGTTGTGGTCCTTCTTCT | 228 | |
| | [symbol] | ATCCAGTTGCCTTCTTGGGACTGA | TAAGCCTCCGACTTGTGAAGTGGT | 134 | |
| | [symbol] | CGAAGCAGAACGATTGCGAG | TGGGAGGCCCTGATGATACA | 349 | |
| | [symbol] | CGCCCTGGCTCCGACTCAGAC | GAGGGTCATGAACCATCACTT | 1061 | |
| | [symbol] | TGGAACCTTCCAGAAGTGGG | TCTTCACACTGAACCCAAGCTC | 433 | |
| | [symbol] | GGAGTATTGGGAGCGGATCAC | GCGTTCCCGTTCTTCAGGTA | [symbol] | |
| Mouse | [symbol] | ATGCTGTGTTTGCCTGGACA | GCTCTGGCCTGATAACTGAGAAT | 781 | |
| | [symbol] | ACTCAGAGATGAGCAAATGCC | CAGGTTGACTGGTAGTTAGTGC | 105 | |
| | [symbol] | AATGGAACTCGGTGAACATCTG | GGGGTTGCTCGACTTTGAC | [symbol] | |
| | [symbol] | TCAGCCTCCTAGCTGTTGG | GTCAACTCTACACTGGTTCCTG | 88 | |
| | [symbol] | AAGAAAATGGACGCCGAACTT | AAGCCATATAGACAACGAAGGTG | 139 | |
| | [symbol] | GTGGGCCGCCCTAGGCACCA | CTCTTTGATGTCACGCACGATTTC | 540 | |
| | [symbol] | | | | SA Biosciences [symbol] |
| | [symbol] | | | | SA Biosciences [symbol] |
| | [symbol] | | | | SA Biosciences [symbol] |
| | [symbol] | | | | SA Biosciences [symbol] |
| | [symbol] | | | | SA Biosciences [symbol] |
| | [symbol] | | | | SA Biosciences [symbol] |
| | [symbol] | | | | SA Biosciences [symbol] |
| | [symbol] | | | | SA Biosciences [symbol] |
| Human | [symbol] | CGCAGTATTGGGAGTGGACC | AGATCCTCCAAAGGCACACGTT | 396 | |
| | [symbol] | GGGGCAAGATGGTAATGAAG | CCAGGATACTGAGGGCATGT | 102 | |
| | [symbol] | GGGTGGTTGTCGTGTGAGTG | GTGTCCCGATTTGACCACAG | 96 | |
| | | CGCGAGAAGATGACCCAGATC | TTGCTGATCCACATCTGCTGG | 731 | |

[symbol]

indicates data missing or illegible when filed

### Example 17

### In Situ Hybridization

Spinal cords were removed from 60 day old wild-type mice and frozen in M1 embedding matrix (Shandon, Pittsburgh). The negative control, labeled with H2-K^{b}/H2-D^{b}-KO, was an H2-K^{b}−/−H2-D^{b}−/− double knockout as previously described (McConnell, M. J., Huang, Y. H., Datwani, A. & Shatz, C. J. H2-K(b) and H2-D(b) regulate cerebellar long-term depression and limit motor learning. Proc Natl Acad Sci USA, 106, 6784-6789 (2009)). Twelve μm cryostat sections were obtained, affixed to slides, air-dried, and stored at −80° C. In situ hybridization was performed as previously described (McConnell, M. J., Huang, Y. H., Datwani, A. & Shatz, C. J. H2-K(b) and H2-D(b) regulate cerebellar long-term depression and limit motor learning. Proc Natl Acad Sci USA, 106, 6784-6789 (2009); Syken, J. & Shatz, C. J. Expression of T cell receptor beta locus in central nervous system neurons. Proc Natl Acad Sci USA, 100, 13048-13053 (2003)). Briefly, slides were thawed and fixed in 4% paraformaldehyde before proteinase K (1 μg/ml) treatment. Slides were then acetylated and dehydrated in an ethanol series (50%, 75%, 2×95%, and 2×100%). Labeled (α-³¹S-UTP) riboprobe was diluted to 0.75×10⁷ cpm/ml in 1×Denhardt's solution with 50% deionized formamide, 10% dextran sulfate, 0.3 M NaCl, 10 mM Tris-HCl pH 8.0, and 1 mM EDTA pH 8.0; applied to sections; and then hybridization took place at 62° C. for 12-18 h. After hybridization, coverslips were floated off in 4×SSC, and then treated with 50 μg/ml RNase A for 30 min at 37° C. Slides were washed with a series of SSC solutions, beginning at 2× and concluding with a high-stringency wash of 0.1×SSC (0.15 M sodium chloride/0.015 M sodium citrate, pH 7) at 60° C. for 30 min. Finally, sections were dehydrated through an ethanol series and placed on film. After exposure to Kodak XAR-5 film at room temperature, sections were coated with NTB-2 emulsion and developed after 2-4 weeks.

The sequence of the H2-D^{b} probe was: 3′-AGGTGGGCTACGTGGACGACGAGGAGTTCGTGCGCTTCGACAGCGACGCGGAGA ATCCGAGATATGAGCCGCGGGCGCCGTGGATGGAGCAGGAGGGGCCGGAGTATT GGGAGCGGGAAACACAGAAAGCCAAGGGCCAAGAGCAGTGGTTCCGAGTGAGC CTGAGGAACCTGCTCGGCTACTACAACCAGAGCGCGGGCGGCTCTCACACACTC CAGCAGATGTCTGGCTGTGACTTGGGGTCGGACTGGCGCCTCCTCCGCGGGTACC TGCAGTTCGCCTATGAAGGCCGCGATTACATCGCCCTGAACGAGAACCCAC-5′ (SEQ ID NO: 7). Adjacent sections were hybridized with sense and antisense probes. No specific hybridization was seen using sense probes.

### Example 18

### Fixation and Immunostaining

Cells were fixed with 4% paraformaldehyde (PFA) for 10 min. Mouse spinal cords were obtained by intracardiac perfusion with 4% PFA followed by 24 hours of post-fixation. Spinal cords were rinsed twice with 0.1 M sodium phosphate buffer and immersed in 30% sucrose for 2 days at 4° C. or until the spinal cords sank to the bottom of the 50 ml conical. Fixed spinal cords were embedded and sectioned using a vibratome (40 μm). For antigen detection using frozen sections, mouse spinal cord tissues were cut in 5- to 6-mm sections and embedded in Tissue-Tek OCT compound (Sakura Finetek) and frozen with dry ice. Tissues were then sectioned at 10 μm with a cryostat and then stored at −20° C. in an anti-freezing solution before immunocytochemical analysis. Paraffin-embedded human spinal cord tissues were obtained from NDRI and from Emory University, GA. A summary of the demographic information associated with the human spinal cord tissues is shown in Table 3.

TABLE 3
Summary of the demographic information associated with
human spinal cord tissues used for immunostaining

| | | | | Time from | | Spinal |
|---|---|---|---|---|---|---|
| | | Age | | Diagnosis | | Cord |
| ID | Diagnosis | (yr.) | Gender | to Death | | Segments |
| 63628 | Non-ALS | 67 | Male | N/A | | Thoracic |
| E09-170 | Non-ALS | 88 | Female | N/A | | Lumbar |
| 4944MA | Familial ALS ^{*} | 57 | Male | 8 | months | Thoracic |
| E10-83 | Familial ALS * | 65 | Female | 4 | months | Lumbar |
| 57746 | Sporadic ALS | 61 | Male | 3 | weeks | Lumbar |
| 63470 | Sporadic ALS | 67 | Male | 1.2 | months | Lumbar |
| 60897 | Sporadic ALS | 62 | Female | N/A | | Cervical |
| E08-67 | Sporadic ALS | 49 | Female | 2.5 | years | Lumbar |
| E08-86 | Sporadic ALS | 71 | Male | 6 | months | Lumbar |
| E08-125 | Sporadic ALS | 55 | Female | 1 | year | Thoracic |

N/A, non-available;
* sequencing results confirmed A4V mutation in the SOD1 locus.

Tissues were sectioned at 10 μm and antigen retrieval methods were applied based on manufacturer's suggestions where primary antibodies were purchased. Staining of control and experimental groups was performed in parallel. Antibodies used are listed in Table 2. For most antigens, samples were first incubated for 1 hour in TBS containing 0.1% triton-X and 10% donkey serum, followed by incubation with the primary antibody for 48-72 hours at 4° C. Labeling with secondary antibodies conjugated with various fluorochromes was performed for 2 hours at room temperature.

MHCI staining was performed according to a previously described protocol, with minor modifications (Nardo, G., et al. Transcriptomic indices of fast and slow disease progression in two mouse models of amyotrophic lateral sclerosis. Brain 136, 3305-3332 (2013); Thams, S., et al. Classical major histocompatibility complex class I molecules in motoneurons: new actors at the neuromuscular junction. J Neurosci 29, 13503-13515 (2009)). The antibody ER-HR52 recognizes histocompatibility 2 subclasses for mouse classical MHCI molecules and the antibody EMR8-5 recognizes all HLA-A, B and C of the human classical MHCI molecules (referred to herein as MHCI). Briefly, for in vitro MHCI labeling, cells on coverslips were fixed, blocked and incubated with primary and secondary antibodies without membrane permeabilization during the staining process. MHCI fluorescence intensity per MN was automatically measured using Adobe Photoshop CS5 extended version (Adobe, San Jose, CA). For in vivo MHCI labelling, cell permeabilization was achieved using 0.05% triton-X for mouse spinal cord samples and 0.1% saponin for human spinal cord samples for 30 minutes at room temperature. Incubation with primary and secondary antibodies was performed in 10% donkey serum without any detergent. Detection of MHCI in paraffin embedded human tissue was achieved with 3,3′-diaminobensidine staining by using the ABC and VectorRed Kit protocols (Vector Laboratories, Burlingame, CA). Tissues were counterstained with Hematoxylin QS solution (Vector Laboratories). Fluorescence images were captured on a laser scanning confocal microscope (Carl Zeiss Microscopy, Thornwood, NY) and 3,3′-diaminobensidine stained images were captured with the Zeiss Axioscope.

TABLE 2
Antibodies and details associated with their use.

| | | Catalog | | Dilution | | Tissue |
|---|---|---|---|---|---|---|
| | Company | Number | Host | Used | Application | Fixation |
| Mouse Specimen | | | | | | |
| LY49C/I | BD Pharmagin | 553273 | Mouse | 1:50 | ICC/IHC | No |
| MHC Class I (ER-HR52) | AbD Serotec | mca2398 | Rat | 1:100 | ICC/IHC | PFA |
| GFAP | Abcam | ab [symbol] 874 | Chicken | 1:500 | ICC/IHC | PFA |
| EAAT2 | Gift from Dr. | | Rabbit | 1:200 | ICC | PFA |
| | Jeffrey Rothstein | | | | | |
| ChAT | Millipore | ab144 [symbol] | Goat | 1:200 | ICC/IHC | PFA |
| GFP | Abcam | ab13970 | Chicken | 1:500 | ICC/IHC | PFA |
| RFP | Abcam | ab34771 | Rabbit | 1:1 [symbol] 00 | ICC | PFA |
| S100 [symbol] | Swant | 37A | Rabbit | 1:500 | ICC | PFA |
| CD [symbol] A | Abcam | ab22378 | | 1:200 | ICC/IHC | No |
| GAD67 | Millipore | MA85 [symbol] 06 | Mouse | 1:200 | ICC | PFA |
| TUJ1 | Covance | PR [symbol] -435P | Rabbit | 1:400 | ICC | PFA |
| Human Specimen | | | | | | |
| KIR3DL2 | Abcam | ab95303 | Rabbit | 1:200 | ICC/IHC | Formalin |
| MHC Class I (EMR8-5) | Abcam | ab70328 | Mouse | 1:50 | IHC | Formalin |
| GFAP | Abcam | ab [symbol] 67 [symbol] | Chicken | 1:500 | ICC/IHC | Formalin |
| ChAT | Millipore | AB143 | Rabbit | 1:200 | ICC | PFA |
| HB [symbol] | Abcam | ab2 [symbol] 128 | Rabbit | 1:250 | ICC | PFA |
| SMI32 | Covance | SMI-32R | Mouse | 1:500 | ICC | PFA |
| GFP | Abcam | ab13 [symbol] 70 | Chicken | 1:500 | ICC | PFA |

ICC, Immunocytochemistry; IHC, Immunohistochemistry; PFA, Paraformaldehyde.
LY49C/I and CD8A staining in tissue was performed in frozen sections. [symbol]
indicates data missing or illegible when filed

### Example 19

### MNs Lose MHCI Expression in ALS

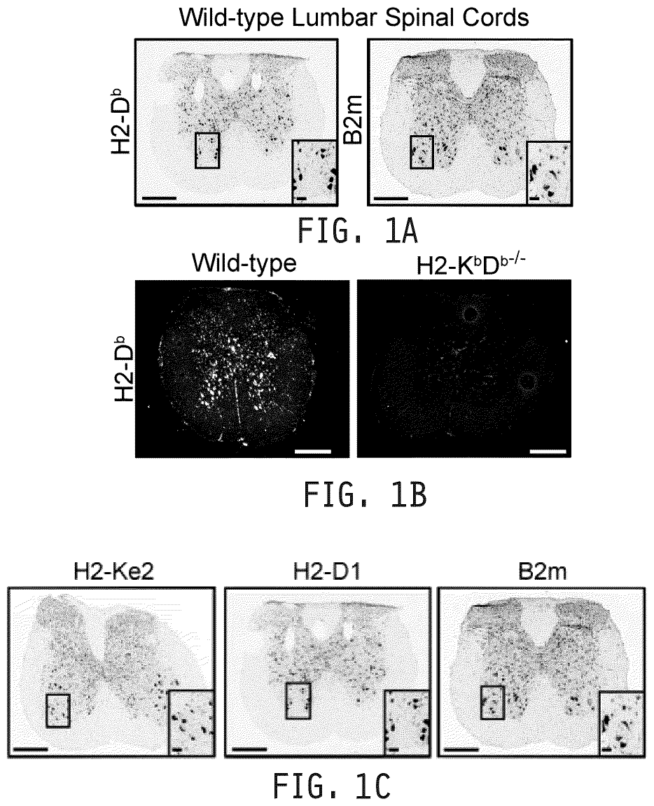

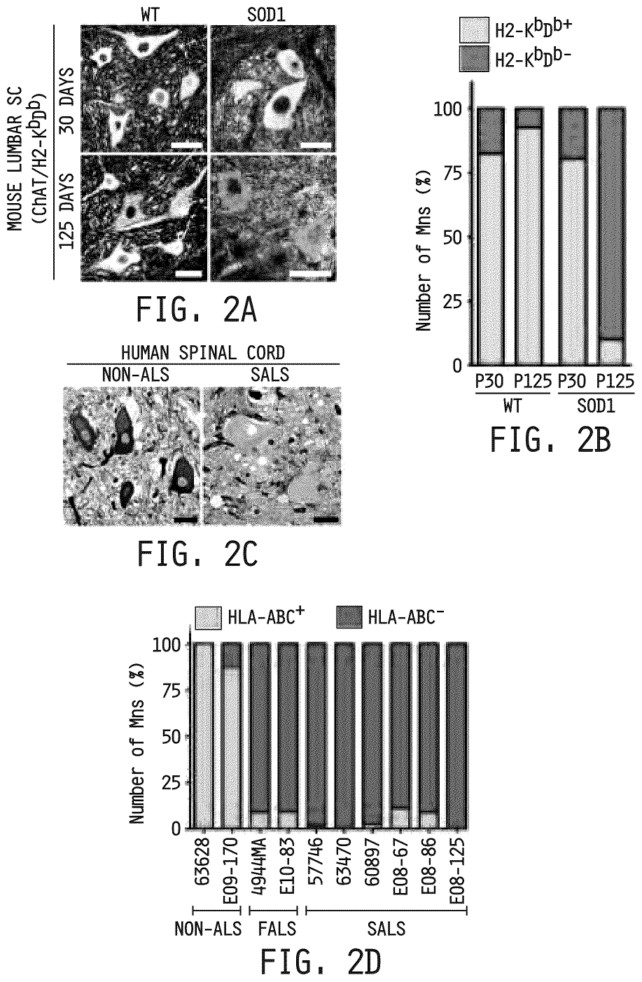

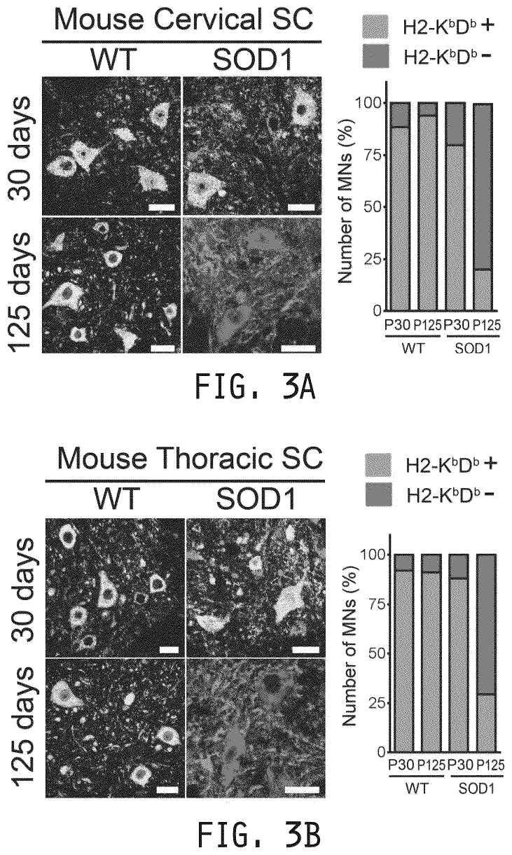

Molecules of the MHCI subclasses are expressed in the adult CNS. MHCI molecules and β2m are enriched in MNs and have been implicated in ALS (FIGS. 1A-1C). In order to examine MHCI expression in MNs in ALS, MHCI expression was analyzed prior to and after disease onset in all segments of the spinal cord of SOD1^{G93A} mice and compared them to wild-type mice. Using an antibody that recognizes subgroups of mouse MHCI called as histocompatibility 2 K and D (H2-K^{b} and H2-D^{b}), significant loss of MHCI expression in the MN somata was observed throughout the entire spinal cord. This loss became specifically evident after disease onset in SOD1^{G93A} mice, while wild-type mice showed robust expression at corresponding time pints (FIGS. 2A, 2B and 3).

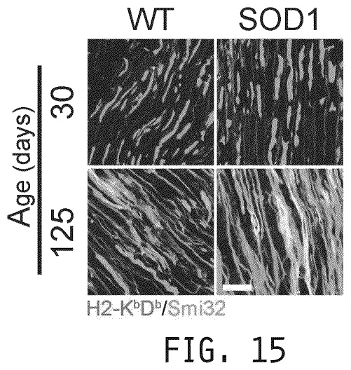

In rapidly progressive SOD1 mouse model (129Sv-SOD1^{G93A}), MHCI protein is transported away from MN cell body and accumulated in peripheral motor axons during disease course (Nardo, G., et al. Transcriptomic indices of fast and slow disease progression in two mouse models of amyotrophic lateral sclerosis. Brain 136, 3305-3332 (2013)). Using fast progressing SOD1 mice (B6SJL-SOD1^{G93A}), motor axons in the sciatic nerves showed increased MHCI immunoreactivity with a marked reduction in MN soma after disease onset (FIG. 15).

To determine if loss of MHCI in MNs seen in the mouse model was also seen in human ALS patients, MHCI expression was evaluated by immunohistochemistry in spinal cord samples from familial ALS (FALS) patients carrying the SOD1^{A4V} mutation and sporadic patient as well as non-ALS controls. An antibody recognizing human MHCI was used; human leukocyte antigen (HLA)-A, -B, and -C. As shown in FIG. 2C and quantified in FIG. 2D, MHCI expression in MNs was almost completely absent in both FALS and SALS spinal cords in agreement with the ALS rodent model, whereas MHCI levels were strong in MNs of non-ALS samples. Taken together, the mouse and human data show that MHCI expression in MNs is diminished following disease onset, with a majority of MNs perikarya showing very low to no expression of MHCI at the later stage of disease.

### Example 20

### Exposure of MNs to ALS Astrocytes Results in Loss of MHCI Expression

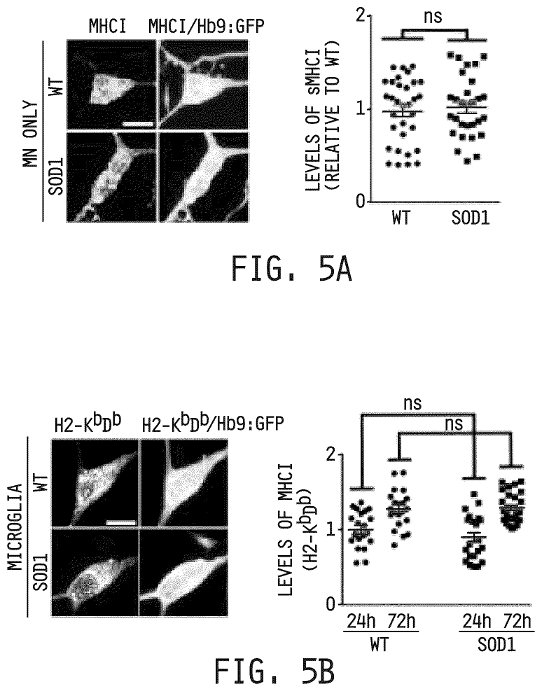

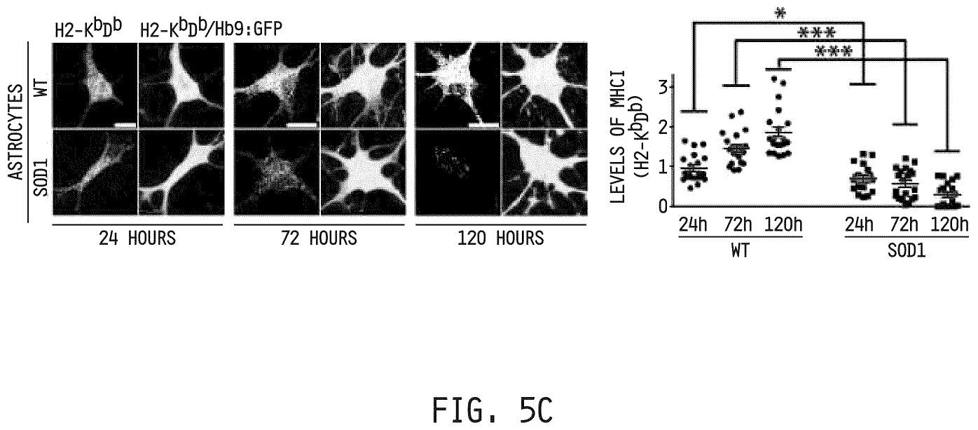

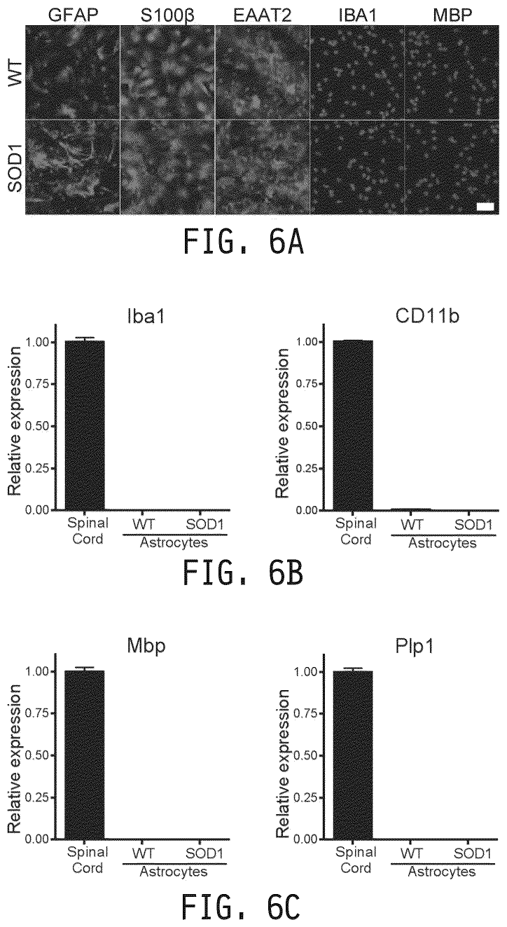

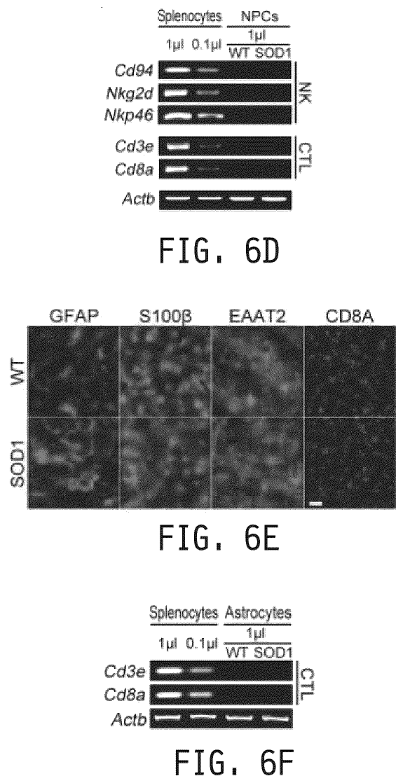

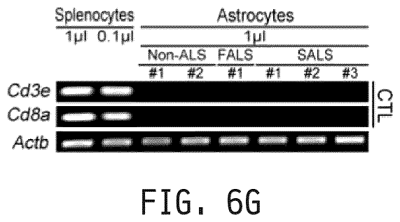

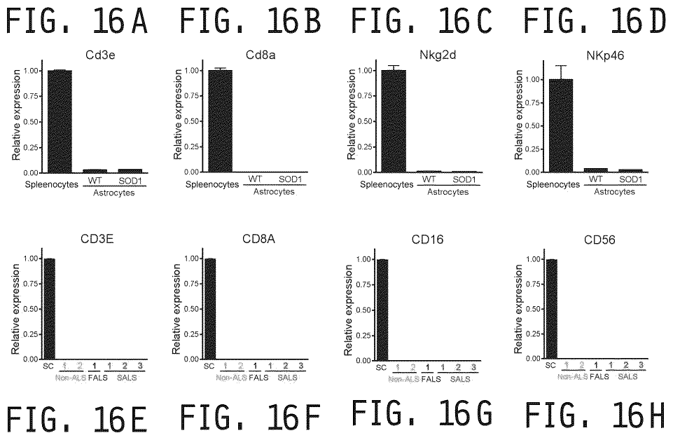

In view of the role of glia cells in MN death (Ilieva, H., et al. Non-cell autonomous toxicity in neurodegenerative disorders: ALS and beyond. J Cell Biol 187, 761-772 (2009); Philips, T. et al. Glial cells in amyotrophic lateral sclerosis. Exp Neurol (2014)), ALS glia were investigated as possible contributors to the loss of MHCI expression in MNs. Using a described co-culture system of adult CNS-derived microglia and MNs (Frakes, A. E., et al. Microglia induce motor neuron death via the classical NFkappaB pathway in amyotrophic lateral sclerosis. Neuron 81, 1009-1023 (2014)), the impact of ALS microglia on the expression of MHCI in MNs was evaluated. SOD1^{G93A} microglia were toxic to MNs. However, as shown in FIG. 5A, when MNs were co-cultured with wild-type or SOD1^{G93A} microglia, no overt changes were observed in MN MHCI levels. When MNs were cultured in the presence of ALS astrocytes, MHCI expression in MNs was diminished. A clear down-regulation of MHCI in MNs was observed within 24 hours in the presence of SOD1^{G93A} astrocytes and levels steadily declined over the next 96 hours, at which point about 73% of MNs completely lost initial MHCI expression. In contrast, MHCI expression in MNs steadily increased during the same period when MNs were cultured on top of wild-type astrocytes (FIG. 5B). This may reflect MN maturation in the presence of astrocytes (Clarke, L. E. et al. Emerging roles of astrocytes in neural circuit development. Nature reviews. Neuroscience 14, 311-321 (2013)) (FIG. 5C), which affects MHCI expression patterns in neurons (Liu, J., et al. The expression pattern of classical MHC class I molecules in the development of mouse central nervous system. Neurochemical research 38, 290-299 (2013)). Astrocytes used in this study were derived from spinal cord neural progenitor cells (NPCs) (Miranda, C. J., et al. Aging brain microenvironment decreases hippocampal neurogenesis through Wnt-mediated survivin signaling. Aging Cell (2012)). These cells were highly enriched for prototypic astrocyte markers, such as glial fibrillary acidic protein (GFAP), S100 calcium binding protein B (S100β), as well as excitatory amino-acidic transporter (EAAT2) without evident contamination by other cell types (FIG. 6A). No detectable microglia or oligodendrocytes were found in the astrocyte cultures as assessed by immunohistochemistry and quantitative RT-PCR (FIG. 6A). The absence of cytotoxic T lymphocytes (CTLs) and natural killer (NK) cells that may affect MHCI expression levels in the cultures was also confirmed by quantitative RT-PCR (FIGS. 16A-16D).

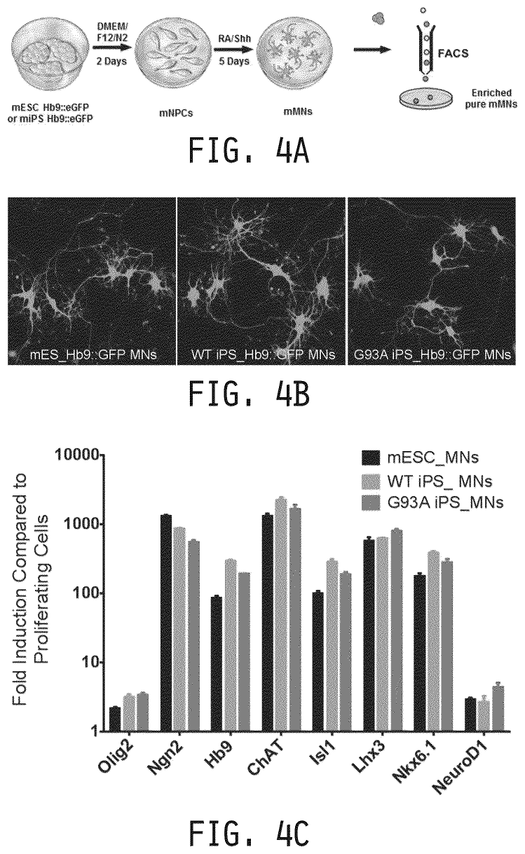

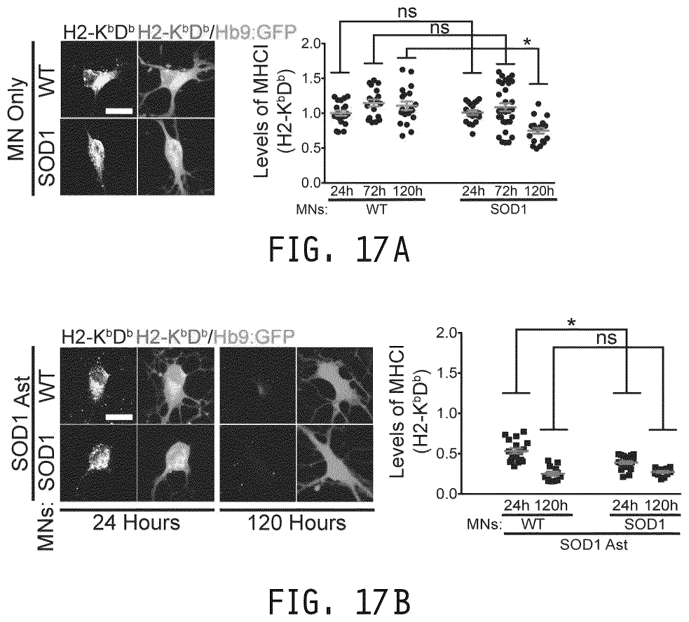

To evaluate if expression of ALS linked-mutant SOD1 protein within MNs could lead to intrinsic down-regulation of MHCI expression, wild-type or SOD1^{G93A} MNs were generated using induced pluripotent stem cell (iPSC) technology (Israelson, A., et al. Macrophage Migration Inhibitory Factor as a Chaperone Inhibiting Accumulation of Misfolded SOD1. Neuron 86, 218-232 (2015)). IPSCs were generated using NPCs expressing the green fluorescent protein (GFP) under the control of the MN specific Hb9 promoter. These iPSCs were differentiated towards MN lineage and sorted by Hb9-GFP expression using a fluorescence activated cell sorter (FIGS. 4A and 4B). Wild-type and SOD1^{G93A} iPSC derived MNs grown in monoculture showed neuronal morphology and gene expression profiles similar to MNs derived from mouse embryonic stem cells (ESCs) (FIG. 4C). As shown in FIG. 17A, there was no significant change in MHCI expression between wild-type and SOD1^{G93A} MNs for the first 72 hours, and only a 27% of MHCI down-regulation was observed specifically in SOD1^{G93A} MNs by 120 hours. Furthermore, mutant SOD1 expressing MNs did not display lower levels of MHCI upon co-culture with SOD1^{G93A} astrocytes compared to wild-type MNs co-cultured with SOD1^{G93A} astrocytes (FIG. 17B), suggesting ALS astrocytes may act as a main contributor for down-regulation of MHCI in MNs.

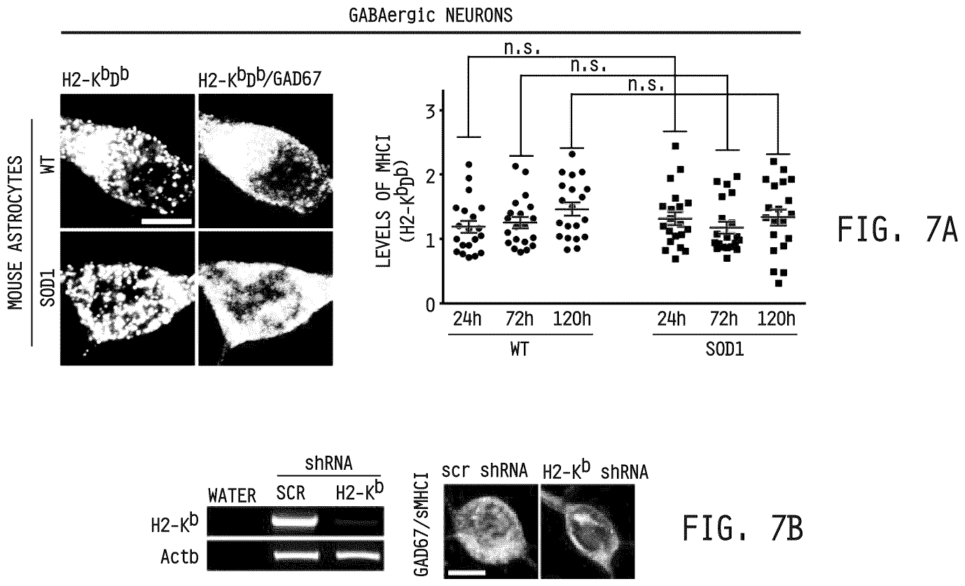

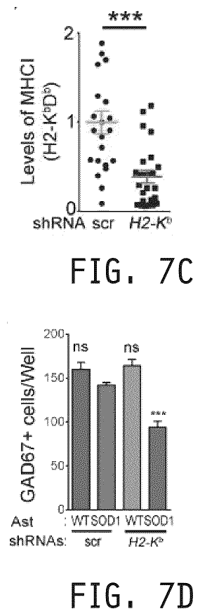

Changes in MHCI expression in GABAergic neurons, a neuronal population spared from ALS astrocyte induced toxicity when co-cultured, was also evaluated (Marchetto, M. C., et al. Non-cell-autonomous effect of human SOD1 G37R astrocytes on motor neurons derived from human embryonic stem cells. Cell Stem Cell 3, 649-657 (2008); Nagai, M., et al. Astrocytes expressing ALS-linked mutated SOD1 release factors selectively toxic to motor neurons. Nature neuroscience 10, 615-622 (2007)). In contrast to MNs, MHCI expression in GABAergic neurons remained constant throughout the culture period in the presence of SOD1^{G93A} astrocytes (FIGS. 7A-7D).

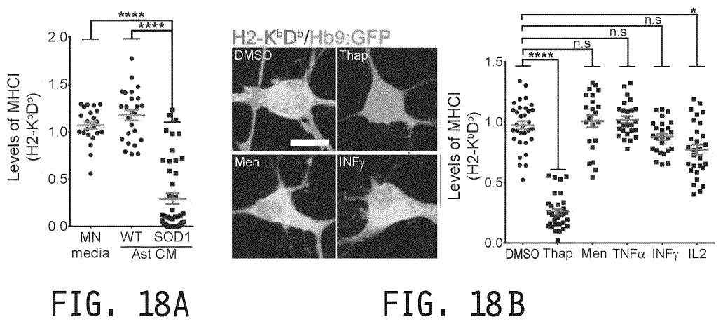

Astrocytes kill MNs not only by cell contacts, but also by the release of soluble factors. To determine whether cell contacts between MNs and astrocytes were required for MHCI loss in MNs, MNs were cultured in the absence of astrocytes, but with medium conditioned by either wild-type or SOD1^{G93A} astrocytes, and the MHCI levels in MNs were measured. As shown in FIG. 18A, when MNs were cultured with SOD1^{G93A} astrocytes conditioned medium, it was found that about 84% of MNs already lost MHCI expression by 24 hours when >95% MNs still survived. This observation strongly suggests ALS astrocyte secrete factors that may lead to a down-regulation of MHCI in MNs. A handful of characterized compounds or recombinant proteins known to be secreted from SOD1^{G93A} astrocytes were tested for their ability to modulate MHCI levels in MNs. The compounds were chosen based on their impact on MN survival pathway, such as endoplasmic reticulum (ER) stress, oxidative stress, and inflammatory response. Since these compounds may greatly impact MN viability, MNs were cultured with these compounds for 9 hours, a period in which no significant signs of MN death were observed. It was found that thapsigargin, a sarco-endoplasmic reticulum calcium ATPase inhibitor that induces ER stress in MNs (Nishitoh, H., et al. ALS-linked mutant SOD1 induces ER stress- and ASK1-dependent motor neuron death by targeting Derlin-1. Genes & development 22, 1451-1464 (2008)) leads to loss of MHCI expression in more than 76% of MNs. Menadione, an oxidative stress inducer, did not lead to a down-regulation of MHCI. The pro-inflammatory molecules TNFα, IFNγ and IL2 showed moderate effects with only about 10% MNs displaying reduced MHCI levels (FIG. 18B). Taken together, these results suggest that astrocytes secrete ER stress inducers to cause loss of MHCI in MNs as a key cellular component in ALS.

### Example 21

### Levels of MHCI Expression in Neuronal Cells Determine their Susceptibility to ALS Astrocyte Induced Toxicity

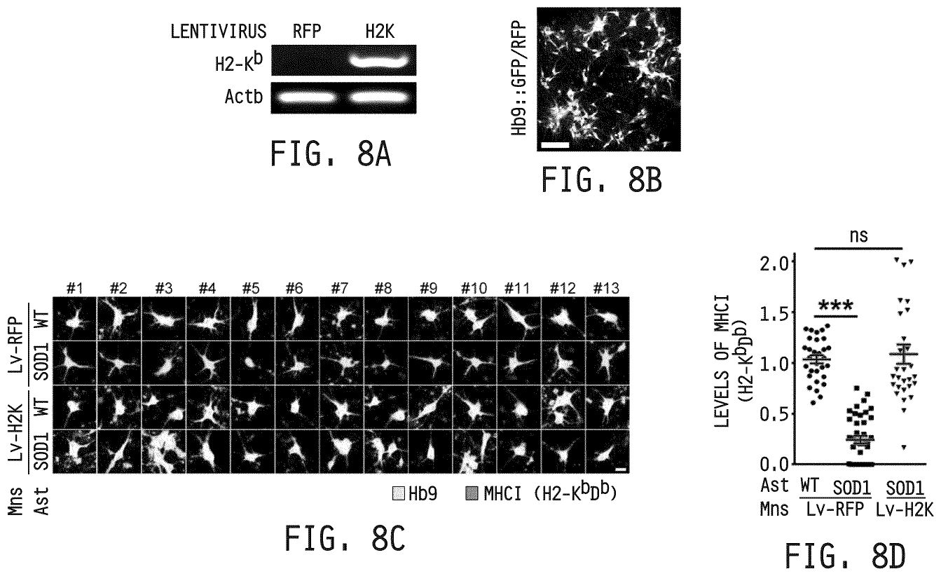

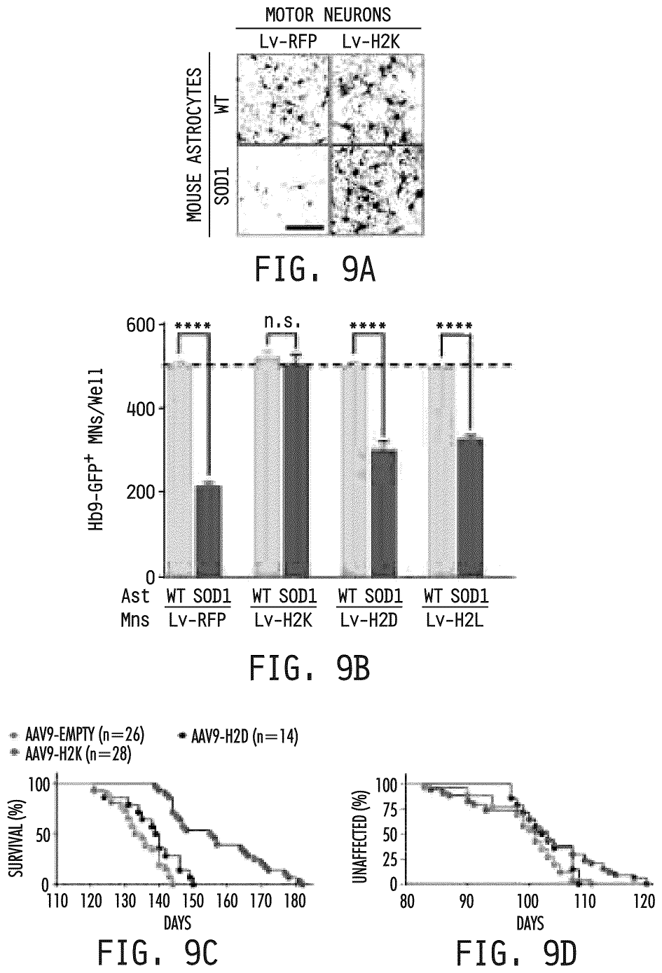

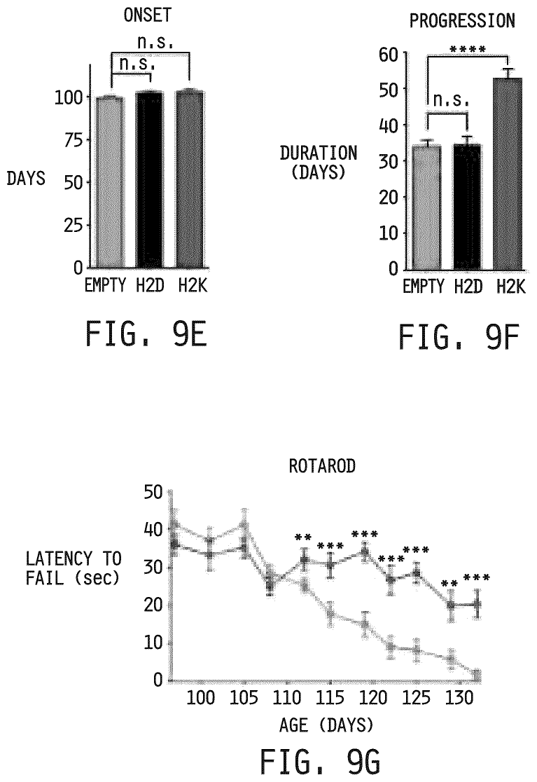

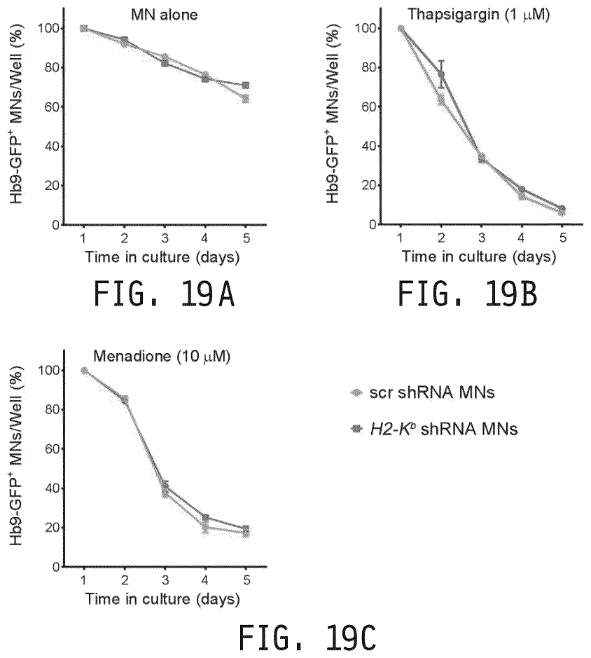

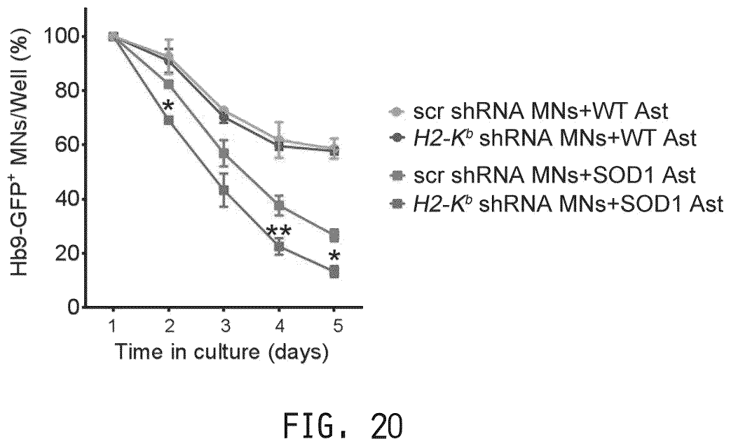

The effects of three MHCI molecules were evaluated by overexpressing them in MNs prior to co-culture with mouse ALS astrocytes and determined MN survival. Mouse classical MHCI subclasses; H2-D^{b}, H2-K^{b} or H2-L^{d} were delivered via lentiviral vectors to Hb9::GFP sorted MNs. Lentiviral transduction resulted in more than 80% MN transduction as shown by the control vector expressing the red fluorescence protein (RFP) (FIG. 8A-8D). While overexpression of H2-D^{b} or H2-L^{d} in MNs resulted in a modest increase of MN survival, overexpression of H2-K^{b} completely protected them from the toxic effects of SOD1^{G93A} astrocytes. In fact, the survival and morphology of H2-K^{b} transduced MNs did not differ from MNs co-cultured with wild-type astrocytes (FIGS. 9A and 9B). In addition to an increase in MN survival, protection evident by unaltered MN soma size and neuritic length was also observed (data not shown). Expression of RFP as a transduction control in MNs did not alter SOD1^{G93A} astrocyte-mediated toxicity, with more than 60% of MNs dying within 120 hours of co-culture (FIGS. 9A and 9B). To further show the observation that H2-K^{b} confers protection to MNs, the effects of H2-K^{b} suppression in MNs were tested by delivering a shRNA. H2-K^{b} suppression in MNs did not lead to intrinsic MN cell death (FIG. 19A). However, upon co-culture with SOD1^{G93A} astrocytes, H2-K^{b} shRNA treated MNs showed reduced survival with a 15.4% increase in cell death by 48 hours, and an even greater cell death (50.2%) by 120 hours when compared to scrambled shRNA transduced MNs (FIG. 20). Suppression of H2-K^{b} did not affect MN survival when co-cultured with wild-type astrocytes (FIG. 20). Moreover, H2-K^{b} suppressed MNs did not show increased susceptibility to other stress molecules (FIGS. 19B and 19C). The effects of H2-K^{b} suppression in GABAergic neurons were also tested. As shown in FIG. 7D, upon co-culture with SOD1^{G93A} astrocytes, GABAergic neurons with reduced MHCI expression showed a significant decrease in survival, with an observed 43.7% cell death compared to scrambled shRNA transduced GABAergic neurons. Taken together, these findings suggest that MHCI expression by neuronal cells determines their susceptibility to ALS astrocytes and sustained expression of MHCI in MNs is sufficient to protect MNs from ALS astrocyte toxicity in vitro.

### Example 22

### Sustained H2-K^{b} Expression in MNs is Associated with Delayed Disease Progression in SOD1^{G93A} Mice

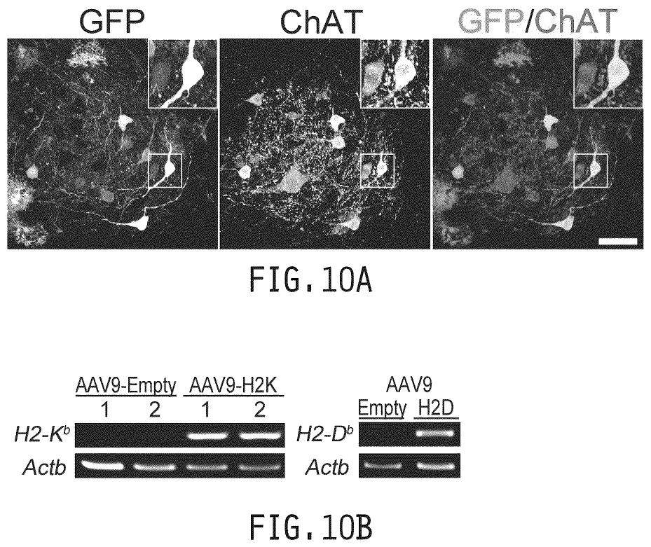

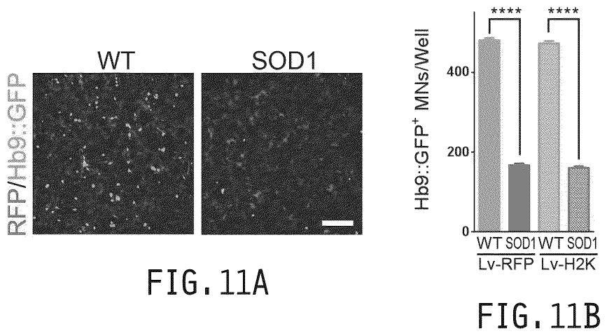

Taking advantage of the ability of AAV9 to readily transduce MNs in the spinal cord when injected in the cerebral spinal fluid (CSF) (Chakrabarty, P., et al. Capsid serotype and timing of injection determines AAV transduction in the neonatal mice brain. PLoS One 8, e67680 (2013); Robbins, K. L., et al. Defining the therapeutic window in a severe animal model of spinal muscular atrophy. Hum Mol Genet 23, 4559-4568 (2014)), MNs in SOD1^{G93A} mice were targeted with AAV9 encoding H2-K^{b} (AAV9-H2K) or H2-D^{b} (AAV9-H2D) under the control of a chicken β-actin promoter. As previously reported and shown in this study, high levels of spinal cord MN transduction in SOD1^{G93A} mice were obtained with injection of AAV9-GFP (FIG. 10A). Delivery of AAV9-H2K (or AAV9-H2D) resulted in a marked increase in H2-K^{b} (or H2-D^{b}) mRNA levels in spinal cords, indicating functionality of our viral construct (FIG. 10B). Expression of H2-K^{b} in MNs via AAV9 delivery at post-natal day 1 in SOD1^{G93A} mice resulted in a 21 day extension in the mean survival of injected SOD1^{G93A} mice compared to control (AAV9-empty) injected SOD1^{G93A} littermates (156.9±2.6 days in AAV9-H2K vs. 135.5±1.6 days in AAV9-empty, unpaired t-test, mean±s.e.m, P<0.0001) (FIG. 9C). 39% of the animals survived over 165 days, with the longest-living mouse reaching 182 days in AAV9-H2K treated animals. When AAV9-H2D was delivered to SOD1^{G93A} mice, no significant changes in the mean survival were observed (139.2±1.4 days in AAV9-H2D vs. 135.5±1.6 days in AAV9-empty, unpaired t-test, mean±s.e.m, P<0.05), consistent with the in vitro observations. Mean disease onset, as assessed by age at the peak body weight, was not significantly altered (103.3±2.0 days in AAV9-H2K vs. 103.1±1.2 days in AAV9-H2D vs. 99.73±1.2 days in AAV9-empty, unpaired t-test, P=0.1) (FIGS. 9D and 9E). However, disease progression was greatly slowed down by ˜50.3% in AAV9-H2K treated mice (52.7±2.6 days in AAV9-H2K vs. 34.1±1.8 days in AAV9-empty, unpaired t-test, P<0.0001), while almost no changes were observed in AAV9-H2D treated mice (34.62±2.2 days in AAV9-H2D vs. 34.1±1.8 days in AAV9-empty, unpaired t-test, P=0.87 (FIG. 9F). Videos taken during the period encompassing prototypic disease progression observed within the SOD1^{G93A} mouse model (from 117 days to 141 days) clearly showed a marked difference between AAV9-H2K injected versus AAV9-empty injected mice (data not shown). During this stage, AAV9-H2K injected SOD1^{G93A} mice showed greater ambulatory capacity compared to AAV9-empty injected animals. Rotarod performance confirmed the significant improvement in motor function observed in AAV9-H2K treated SOD1^{G93A} mice when compared with age-matched controls (unpaired t-test, from P115 to P132, P<0.01 or lower) (FIG. 9G). The delay in disease progression observed when SOD1^{G93A} mice were treated with AAV9-H2K is likely not derived from H2-K^{b} expression in astrocytes since the overexpression of H2-K^{b} in SOD1^{G93A} astrocytes in vitro did not modify their toxicity towards MNs (FIGS. 11A and 11B). These in vivo data support MHCI as a therapeutic molecule that has the potential to significantly delay disease progression in ALS.

### Example 23

### Mouse and Human ALS Astrocytes Express MHCI Inhibitory Receptors

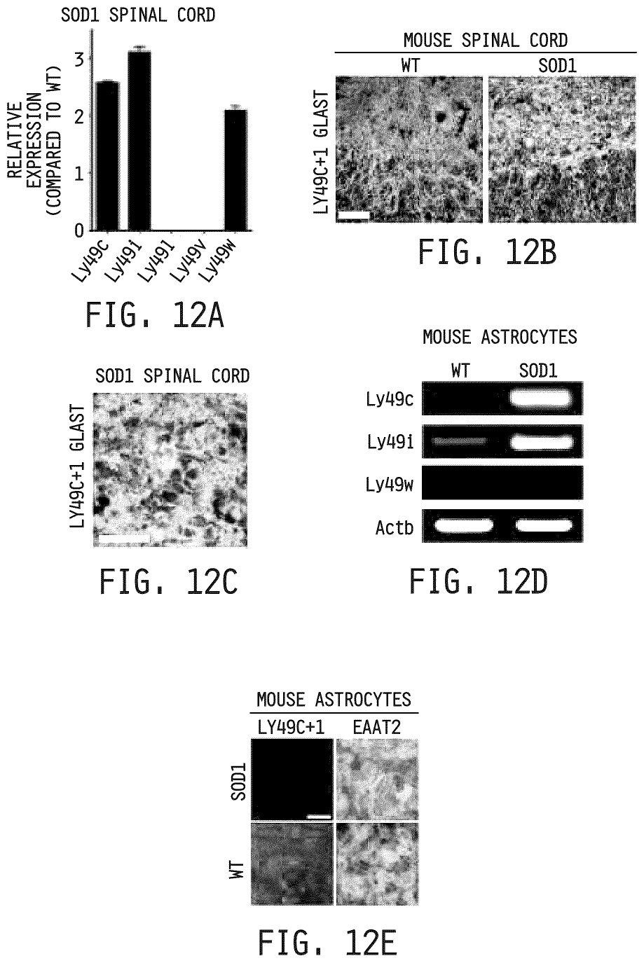

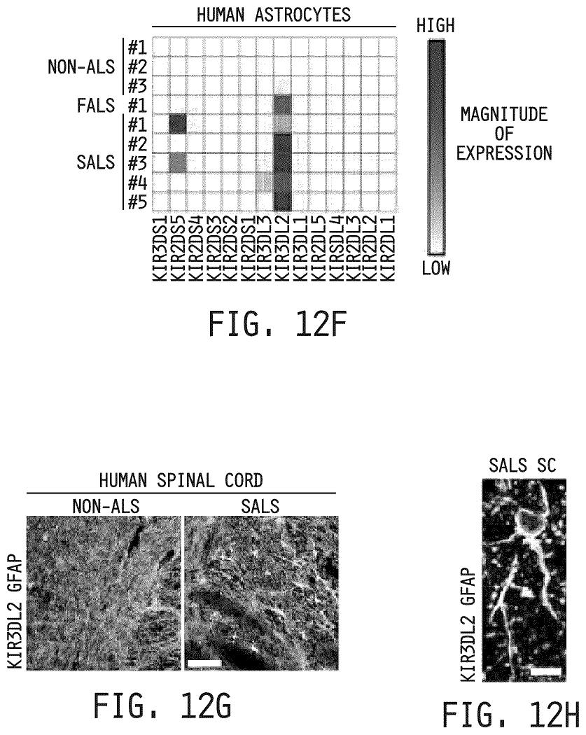

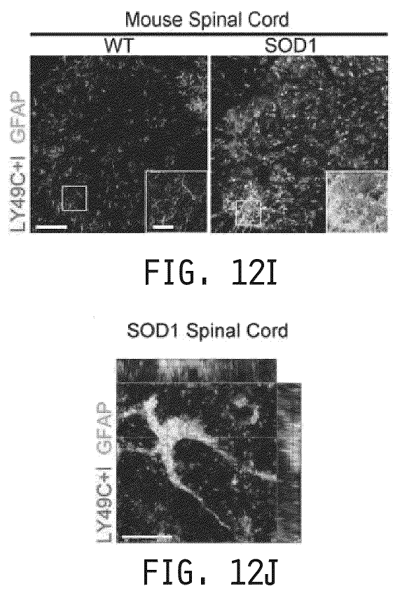

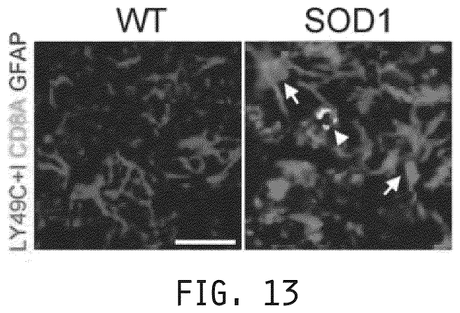

In order for ALS astrocytes to sense reduced levels of MHCI expression in MNs to recognize them as their targets, they should express receptors that can recognize MHCI. An investigation as to how sustained expression of H2-K^{b} in MNs can protect them from SOD1^{G93A} astrocyte mediated toxicity was made. MHCI levels can be a determinant for innate immune cells, particularly natural killer (NK) cells in order to effectively distinguish target cells from healthy cells (Tay, C. H., et al. Control of infections by NK cells. Current topics in microbiology and immunology 230, 193-220 (1998)). Reduced presentation of MHCI antigen on target cells acts as a trigger for cytotoxic lymphocytes to secrete effector molecules and kill the target cells (Lanier, L. L. NK cell recognition. Annual review of immunology 23, 225-274 (2005)). However, when target cell sustained MHCI expression, cytotoxic lymphocytes can sense MHCI using their MHCI receptors. MHCI antigen and receptor interaction results in a signaling cascade in cytotoxic cells, leading to an inhibition of toxicity and survival of target cells (Long, E. O. Regulation of immune responses through inhibitory receptors. Annual review of immunology 17, 875-904 (1999)). To determine if ALS astrocytes had acquired the ability to sense MHCI levels on MNs, expression of MHCI receptors in astrocytes was checked. mRNA analyses were performed for the expression of H2-K receptors in spinal cords of SOD1^{G93A} mice. Ly49c, Ly49i and Ly49w receptors, which are known as H2-K inhibitory receptors were found to be highly expressed in SOD1^{G93A} mice at end stage of disease (FIG. 12A). Immunostaining analysis using antibodies to detect LY49C and LY49I (LY49C/I) receptors confirmed expression of these two receptors in the ventral horn of the lumbar spinal cord of SOD1^{G93A} mice, with little to no expression in age-matched wild-type mice (FIG. 12B). In the lumbar spinal cords of SOD1^{G93A} mice, astrocytes were the prominent cell type expressing LY49C/I receptors as 96±2% of LY49C/I positive cells defined by immunoreactivity to the astrocyte specific membrane protein GLAST or the cytoplasmic protein GFAP (FIGS. 12A and 12C). Furthermore, LY49C/I receptors were found to be highly expressed in SOD1 astrocytes used for the in vitro studies (FIGS. 12D, and 12E). In addition, these receptors were also detected in infiltrating cytolytic T-lymphocytes (CTLs) found in the spinal cord of SOD1^{G93A} mice; however CTL numbers were minimal and therefore only accounted for a small fraction of cells expressing LY49C/I receptors (FIG. 12B). Human ALS patient derived astrocytes were studied to determine if they also express MHCI receptors. RNA expression of a wide panel of 14 MHCI receptors was evaluated. The expression of MHCI inhibitory receptor in human ALS astrocytes, with killer cell immunoglobulin-like receptor 3DL2 (KIR3DL2) was found to be uniquely expressed in all human ALS astrocyte lines tested (FIG. 12F). There was no detectable expression of MHCI inhibitory receptor including KIR3DL2 or any other KIR in non-ALS control astrocyte lines tested. Using immunohistochemical analysis, expression of KIR3DL2 was also confirmed in post-mortem spinal cord samples of SALS patients, where KIR3DL2 expression was predominantly localized to GFAP positive astrocytes (FIGS. 12G and 12H). In summary, these data demonstrate that ALS astrocytes express receptors that can act as sensors for the levels of MHCI of surrounding cells and this cell-to-cell recognition system may be involved in initiating astrocyte mediated MN toxicity.

### Example 24

### HLA-F Protects Human MNs from FALS and SALS Astrocyte Induced Toxicity

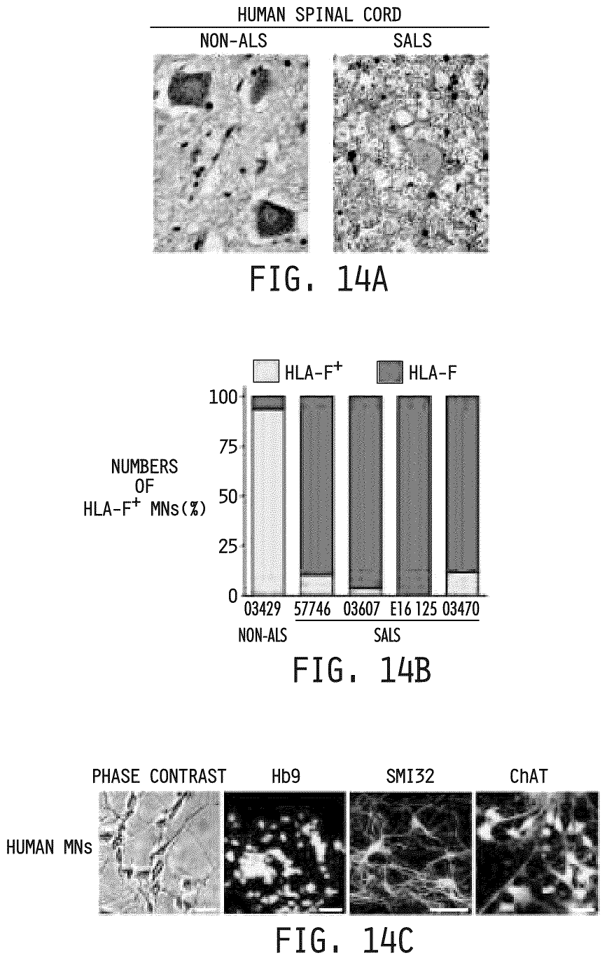

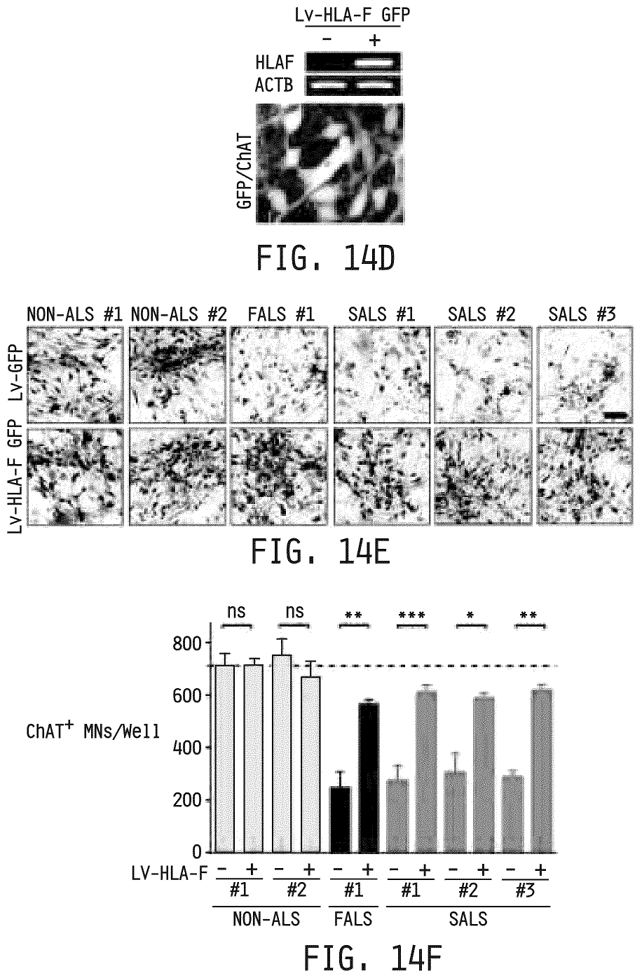

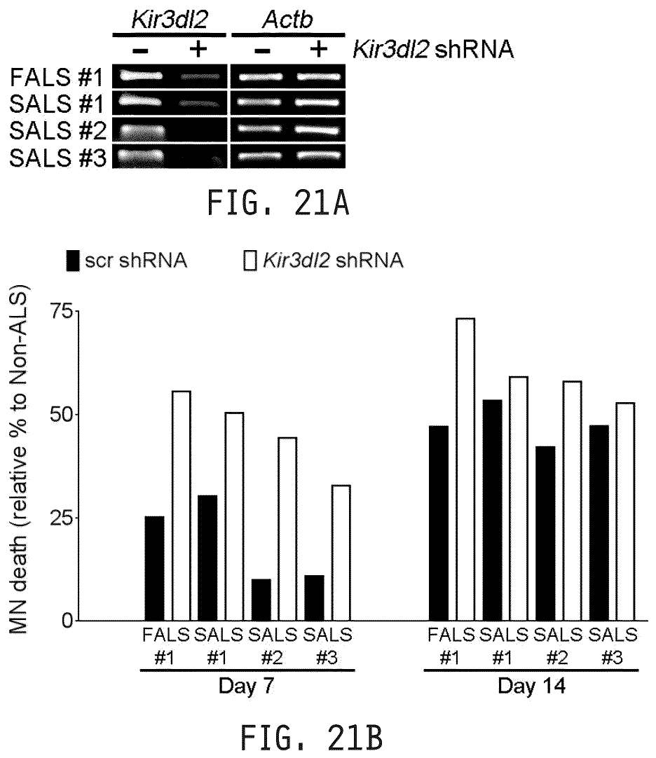

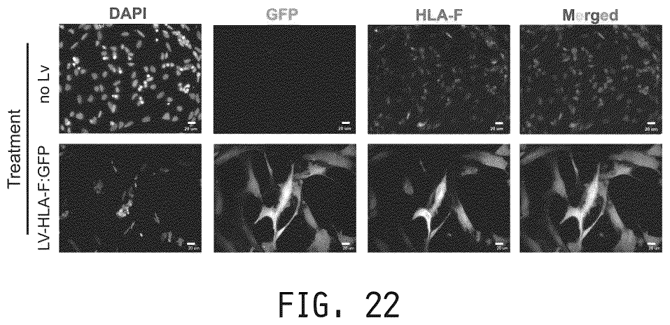

In view of the finding that MHCI receptors expressed in mouse SOD1^{G93A} astrocytes can sense MHCI levels on MNs, leading to inhibition of SOD1^{G93A} astrocyte toxicity, and that human ALS astrocytes also express MHCI receptors, the ability of MHCI molecules to block ALS astrocyte toxicity in a humanized co-culture system was tested. It was hypothesized that sustained expression of human MHCI that is known to bind to the KIR3DL2 receptor will inhibit human ALS astrocyte toxicity towards human MNs. Recently, HLA-F, a human MHCI molecule, was identified as a ligand that can physically and functionally interact with the KIR3DL2 receptor (Hester, M. E., et al. Rapid and efficient generation of functional motor neurons from human pluripotent stem cells using gene delivered transcription factor codes. Molecular therapy: the journal of the American Society of Gene Therapy 19, 1905-1912 (2011)). Since KIR3DL2 was found to be expressed in all FALS and SALS astrocyte lines tested, sustained expression of HLA-F in human MNs may be effective in protecting MNs from ALS astrocyte induced toxicity, regardless of disease etiology. First, a test was performed to determine if HLA-F is expressed in human spinal cord MNs and whether its expression differs between ALS and non-ALS samples. As shown in FIG. 12A, HLA-F is expressed in MNs of non-ALS spinal cord samples. Furthermore, HLA-F expression was dramatically reduced in ALS MNs (FIGS. 14A and 14B) in agreement with findings that overall MHCI expression is reduced in ALS MNs (FIGS. 5C and 5D). Next, the hypothesis that sustained expression of HLA-F in human MNs protects them from ALS astrocyte induced toxicity was tested. To this end, an in vitro model system in which human MNs and human astrocytes were co-cultured (Re, D. B., et al. Necroptosis drives motor neuron death in models of both sporadic and familial ALS. Neuron 81, 1001-1008 (2014)), and cell death was quantified, was implemented. In this system, MNs generated from human embryonic stem cells were instructed to differentiate to a MN lineage using a combination of differentiation molecules and expression of the transcription factors; Ngn2, Isl1 and Lhx3 (Hester, M. E., et al. Rapid and efficient generation of functional motor neurons from human pluripotent stem cells using gene delivered transcription factor codes. Molecular therapy: the journal of the American Society of Gene Therapy 19, 1905-1912 (2011)). Human ESC derived MNs showed neuronal morphology with high levels of the prototypic MN markers; homeobox gene (HB9), neurofilament marker (SMI32) and choline acetyltransferase (ChAT). MN cultures had minimal to no non-neuronal cell contamination (FIG. 14A). In order to transduce MNs, a lentiviral vector encoding human HLA-F cDNA along with an IRES eGFP (Lv-HLAF-IRES-eGFP) was developed to track transduced cells. Transgene expression and high levels of MN transduction were confirmed by HLA-F RNA analysis (FIG. 14B, upper panel) and eGFP visualization upon transduction with Lv-HLAF-IRES-eGFP (efficiency over 90%, FIG. 14B, lower panel). Furthermore, upon Lv-HLA-F:GFP transduction, all cells expressing GFP were found to express HLA-F (FIG. 22). Three-days after the MNs were transduced with Lv-HLAF-IRES-eGFP, astrocytes were added and co-cultured. Human astrocyte lines tested were devoid of a contamination by other glia¹³ and cytotoxic lymphocytes (FIGS. 16E-16H). After 2 weeks of co-culture, MN survival was evaluated by counting cells positive for prototypic MN marker ChAT. Overexpression of HLA-F in human derived MNs resulted in a significant increase in MN survival upon co-culture with either FALS or SALS astrocytes (FIGS. 14C and 14D). No overt change in MN counts was observed when MNs overexpressing HLA-F were co-cultured with non-ALS astrocytes, suggesting a specific effect of HLA-F in preventing ALS astrocyte mediated toxicity. To further evaluate the functional relevance of KIR3DL2 in ALS astrocyte-mediated MN toxicity, a study was made to determine whether suppression of KIR3DL2 in ALS astrocytes will enhance their toxicity towards MNs. When KIR3DL2 is suppressed in ALS astrocytes, astrocytes will lose their ability to sense MHCI antigen presented on MNs. The lack of interaction between MHCI receptor (KIR3DL2) and ligand (MHCI) will not cascade the signaling events that are required to inhibit cytotoxicity and thus, target cells will die. For this purpose, shRNAs against the kir3dl2 gene were generated and tested for this efficiency in knocking down KIR3DL2 expression in human astrocytes (FIG. 21A). As expected, 7 days into the co-culture period, all ALS astrocytes treated with scrambled shRNA were toxic to MNs at various levels depending on the astrocyte line (25.3% with FALS, 30.3% with SALS1, 10.1% with SALS2 and 11.1% with SALS3 compared to non-ALS). By 14 days of co-culture, all astrocyte lines showed similar level of MN death (˜50% compared to non-ALS) (FIGS. 14C, 14D and 21B). When the same MNs were co-cultured with kir3dl2 shRNA treated ALS astrocytes, it was found that a ˜50% MNs death already occurred by day 7. This is a level of cell death that was observed at day 14 when astrocytic KIR3DL2 is not suppressed (FIG. 21B). Therefore, targeting KIR3DL2 in ALS astrocytes promotes toxicity as previously reported in innate immune cells (Bouaziz, J. D., et al. Circulating natural killer lymphocytes are potential cytotoxic effectors against autologous malignant cells in sezary syndrome patients. The Journal of investigative dermatology 125, 1273-1278 (2005).), leading to more rapid MN death. Taken together, these results corroborate the findings in the SOD1^{G93A} mouse model that ALS astrocytes utilize cell-to-cell recognition mechanism in determining target MNs, and indicate that a single MHCI molecule, HLA-F, can protect MNs from both FALS and SALS astrocyte-induced toxicity, a pre-requisite for delaying MN death due to astrocyte toxicity in a broad ALS patient population.

### Example 25

### Statistical Analysis

Statistical analysis was performed under Graph Pad Prism 6 software (La Jolla). Depending on the number of variables and time-points in each experiment, statistical analysis of mean differences between groups was performed by either Student's t-test or multiway ANOVA followed by a Bonferroni post hoc analysis. Kaplan-Meier survival analyses were analyzed by the log-rank test. Comparison of mean survival, disease onset and progression were analyzed by the unpaired t test. Specific statistical tests, P values and sample size are indicated in figure legends.

1. A method for treating amyotrophic lateral sclerosis by increasing HLA-F expression in motor neurons of a patient, the method comprising the step of
administering to the patient a composition comprising an effective amount of a compound that increases the expression of HLA-F in the motor neurons of the patient.

2-48. (canceled)
